(19) United States
(12) Patent Application Publication
(10) Pub. No.: US 2026/0151531 A1
(43) Pub. Date: Jun. 4, 2026

(54) COMPOSITIONS COMPRISING DRAGLINE SPIDER SILK

(71) Applicant: SEEVIX MATERIAL SCIENCES LTD., Jerusalem (IL)

(72) Inventors: Shmuel ITTAH, Mevaseret Zion (IL); Alon MEIR, Jerusalem (IL); Haim Meir GILAD, Sde Boaz (IL)

(73) Assignee: SEEVIX MATERIAL SCIENCES LTD., Jerusalem (IL)

(21) Appl. No.: 19/459,367

(22) Filed: Jan. 26, 2026

Related U.S. Application Data
(62) Division of application No. 17/910,822.

(51) Int. Cl. A61L 17/12; A61L 27/18; A61L 27/22; A61L 27/56; C07K 14/435

(52) U.S. Cl. CPC A61L 17/12; A61L 27/18; A61L 27/227; A61L 27/56; C07K 14/43518

(57) ABSTRACT

Compositions comprising at least one major ampullate spidroin protein (MaSp)-based fiber, a polymer bound to the MaSp-based fiber, and optionally a further polymer having a molecular weight in the range of 1000 Da to 1000 kDa, are provided. Further, methods for preparation of same are provided.

### CROSS REFERENCE TO RELATED APPLICATIONS

This application is a divisional of U.S. patent application Ser. No. 17/910,822, filed Sep. 11, 2022, which is a National Phase of PCT Patent Application No. PCT/IL2020/050286 having International filing date of Mar. 11, 2020, entitled “COMPOSITIONS COMPRISING DRAGLINE SPIDER SILK”, the contents of which are all incorporated herein by reference in their entirety.

### REFERENCE TO AN ELECTRONIC SEQUENCE LISTING

The contents of the electronic sequence listing (SVX-P-006-US1.xml; size: 25,208 bytes; and date of creation: Jan. 26, 2026) is herein incorporated by reference in its entirety.

### FIELD OF THE INVENTION

The present invention, in some embodiments thereof, is directed to compositions comprising fibers made of proteins derived from a MaSp (masegjor ampullate spidroin) protein, and the preparation of same.

### BACKGROUND OF THE INVENTION

Dragline spider silk is known in the art as the silk used by the orb-web weaving spiders to construct the frame and radii of their webs as well a life line when they fall or escape danger. To be able to perform these tasks, the dragline fiber displays a remarkably high toughness due to combination of high elasticity and strength, which places it as the toughest fiber, whether natural or man-made. For instance, dragline is six times as strong as high-tensile steel in its diameter and three times tougher than Kevlar that is one of the strongest synthetic fibers ever made.

Dragline silk consists of two main polypeptides, mostly referred to as major ampullate spidroin (MaSp) 1 and 2, and also to ADF-3 and ADF-4 in Araneus diadematus. These proteins have apparent molecular masses in the range of 200-720 kDa, depending on sample age and conditions of analysis. The known dragline silk spidroins are composed of highly iterated blocks of alternating alanine-rich segments, forming crystalline β-sheets in the fiber, and glycine-rich segments which are more flexible and mainly lack ordered structure. The C-terminal region is non-repetitive, highly conserved between species, and adopts α-helical conformation. The N-terminal region of dragline silk proteins was also found to be highly conserved between different spidroins, and also between different spider species.

Numerous attempts have been made to synthetically create spider silk, such as through genetic engineering using bacteria, yeast, plants and mammalian cells in tissue culture and even transgenic goats.

There is an unmet need for improved compositions and methods for producing fibers with mechanical properties similar to the natural spider silk.

### SUMMARY OF THE INVENTION

According to one aspect of the invention, there is a composition comprising:
a. a porous major ampullate spidroin protein (MaSp)-based fiber;
b. a first polymer bound to the MaSp-based fiber; and
c. a second polymer having a molecular weight in the range of 1000 Da to 1000 kDa.

In one embodiment, bound is via a non-covalent bond, a physical interaction or both.

In one embodiment, the pores of the MaSp-based fiber are partially filled with the first polymer. In one embodiment, the first polymer fills 50% to 100% of the volume of the pores.

In another aspect, there is a composition comprising a copolymer comprising a porous ampullate spidroin protein (MaSp)-based fiber covalently bound to a first polymer. In one embodiment, the copolymer is in a form of a graft-copolymer. In one embodiment, bound is via an amino acid of the MaSp-based fiber. In one embodiment, the amino acid is selected from the group consisting of tyrosine, serine, lysine, and cysteine or any combination thereof. In one embodiment, the composition further comprises a second polymer having a molecular weight in the range of 1000 Da to 1000 kDa.

In one embodiment, the second polymer comprises a partially branched polymer.

In one embodiment, a weight per weight (w/w) ratio of the first polymer to the second polymer within the composition is between 1:7 and 1:30. In one embodiment, the MaSp-based fiber bound to the first polymer is present at a concentration of 0.1% to 30%, by total weight of the composition.

In one embodiment, the first polymer and the second polymer are each independently selected from a synthetic polymer, a thermoplastic polymer, a thermoset, an epoxy, a polyester a polyamide, a polyol, a polyurethane, polyethylene, Nylon, a polyacrylate, a polycarbonate, polylactic acid (PLA) or a copolymer thereof a silicon, a liquid crystal polymer, a maleic anhydride grafted polypropylene, polycaprolactone (PCL), rubber, cellulose, or any combination thereof.

In one embodiment, a w/w ratio of the first polymer to the MaSp-based fiber is between 10:1 and 1:1.

In one embodiment, the first polymer is biodegradable. In one embodiment, the first polymer comprises PCL.

In one embodiment, the first polymer has a molecular weight in the range of 100 Da to 1000 kDa. In one embodiment, the MaSp-based fiber is present at a concentration of 0.1% to 30%, by total weight.

In one embodiment, the MaSp-based fiber comprises a mixture of proteins comprising “m” types of proteins of differing molecular weight, wherein each protein in the mixture comprises, independently, “n” repeats of a repetitive region of a MaSp protein, or a functional homolog, variant, derivative or fragment thereof, wherein m and n are, independently, an integer between 2 to 70.

In one embodiment, the mixture of proteins is characterized by one or more properties selected from the group consisting of: a. each repeat has a molecular weight in the range of 2 kDa to 3.5 kDa; b. the ratio of‘n’ to ‘m’ is in the range of 1.5:1 to 1:1.5.

In one embodiment, each of the proteins comprise, independently, an amino acid sequence as set forth in SEQ ID NO: 1:

(X₁)_{Z}X₂GPGGYGPX₃X₄X₅GPX₆GX₇GGX₈GPGGPGX₉X₁₀,

wherein X₁ is, independently, at each instance A or G wherein at least 50% of (X₁)Z is A, Z is an integer between 5 to 30; X₂ is S or G; X₃ is G or E; X₄ is G, S or N; X₅ is Q or Y; X₆ is G or S; X₇ is P or R; X₈ is Y or Q; X₉ is G or S; and X₁₀ is S or G.

In one embodiment, the repetitive region comprises the amino acid sequence as set forth in SEQ ID NO: 3 (AAAAAAAASGPGGYGPGSQGPSGPGGYGPGGPGSS).

In one embodiment, the MaSp-based fiber is characterized by a porosity of at least 30%. In one embodiment, the MaSp-based fiber is characterized by a porosity of at least 30%.

In one embodiment, the composition further comprises a third polymer. In one embodiment, a w/w content of the third polymer is 30% to 99% of the total composition.

In one embodiment, the composition is in a form of a thread, a sheet or a film.

In another aspect, there is an article comprising the composition of the invention. In one embodiment, the article comprises a biocompatible material. In one embodiment, the article comprises a coating. In one embodiment, the article is in a form of a suture, surgical mesh, medical adhesive strips, electrospun mesh, skin grafts, fat grafts, cosmetics, dermal fillers, drug eluting/delivery device, replacement ligaments, clothing fabric, bullet-proof vest lining, cable, tube, film, rope, fishing line, tires, sports equipment, and reinforced plastics. In one embodiment, the article is in a form of a surgical suture.

In one embodiment, the article comprises the second polymer enriched with the MaSp-based fiber bound to the first polymer.

In one embodiment, the first polymer and the second polymer comprise PCL.

In one embodiment, enriched is between 0.1% and 30%, by total weight of the article.

In one embodiment, the article has a Young's modulus of at least 600 MPa.

In another aspect, there is a medical device comprising the composition of the invention.

In another aspect, there is a method comprising mixing a major ampullate spidroin protein (MaSp)-based fiber with a monomer under conditions suitable for the monomer to polymerize, thereby forming an in-situ polymerized first polymer bound to the MaSp-based fiber.

In one embodiment, bound is via a covalent bond, a non-covalent bond, a physical interaction or a combination thereof.

In one embodiment, polymerize is via a ring-opening polymerization.

In one embodiment, the method is for forming a copolymer comprising the first polymer covalently bound to an amino acid of the MaSp.

In one embodiment, conditions comprise a time period between 1 hour and 4 days and a temperature between 2° and 200° C.

In one embodiment, a w/w ratio of the monomer to the MaSp-based fiber is between 1:1 and 10:1.

In one embodiment, the method further comprises mixing the first polymer bound to the MaSp-based fiber with a second polymer.

In one embodiment, the method is for enriching the second polymer.

In one embodiment, a w/w ratio of the second polymer to the first polymer bound to the MaSp-based fiber is between 100:1 and 5:1.

In one embodiment, the first polymer and the second polymer are each independently selected from a synthetic polymer, a thermoplastic polymer, a thermoset an epoxy, a polyester, a polypropylene, a polyethylene, a polyamide, a polyol, a polyurethane, polyethylene, Nylon, a polyacrylate, a polycarbonate, polylactic acid (PLA) or a copolymer thereof a silicon, a liquid crystal polymer, a maleic anhydride grafted polypropylene, polycaprolactone (PCL), rubber, cellulose, or any combination thereof.

In one embodiment, the monomer comprises a lactone.

In one embodiment, the method is for manufacturing the composition of the invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a bar graph representing Young's modulus enhancement of the enriched polymers versus control (non-treated PCL). PCL was enriched with 3, 6, 10 and 12% w/w of major ampullate spidroin protein (MaSp)-based fiber bound to PCL (SVX-PCL).

FIG. 2 represents strain-stress curves of the enriched polymers (PCL enriched with 3, 6, 10 and 12% w/w of SVX-PCL, versus non-treated PCL as a control).

FIG. 3 is a graph representing ATR-FTIR spectra of SVX fibers (Q) and SVX fibers copolymerized with PCL (A). The additional peak at ˜1730 cm¹ belongs to ester groups of PCL, and confirms a covalent bond between PCL and MaSp-based fibers here is due to the bonding of PCL with SVX fibers.

FIGS. 4A-B represent mechanical properties of the enriched polymeric fibers (PCL fibers enriched with about 10% w/w of SVX-PCL), versus MONOMAX® as a control. FIG. 4A shows comparative strain-stress curves of the SVX-PCL enriched fibers (Δ, n=3) and of MONOMAX® (∘, n=5) as a control. FIG. 4B is a bar graph showing Young's modulus of the SVX-PCL enriched fibers (Δ) and of MONOMAX® (∘) as a control. Data are means; p-value>0.05. SVX-PCL enriched polymeric fibers show increased (about 100%) stiffness (Young's modulus) as compared to MONOMAX®.

FIGS. 5A-B represent knot strength of the enriched polymeric fibers (PCL fibers enriched with about 10% w/w of SVX-PCL), versus MONOMAX® as a control. FIG. 5A shows strain-stress curves of the SVX-PCL enriched fibers (Δ, n=4) and of MONOMAX® (∘, n=3) as a control. Stress development with increasing strain was measured upon formation of a knotted suture (knot was generated in accordance with USP monograph for Absorbable sutures). FIG. 5B shows a bar graph representing knot strength (normalized to suture diameter) of the SVX-PCL enriched fibers (Δ, n=5), versus MONOMAX® (∘, n=3) as a control. SVX-PCL enriched polymeric fibers show increased knot strength (about 50% increase) as compared to MONOMAX®. The normalized knot strength values were obtained as described in FIG. 5A. Data are means; p-value>0.05.

FIGS. 6A-C represent mechanical hysteresis of the enriched polymeric fibers (PCL fibers enriched with about 10% w/w of SVX-PCL), versus MONOMAX® as a control. FIG. 6A represents hysteresis curves of the SVX-PCL enriched fibers (Δ) and of MONOMAX® (∘) as a control. Hysteresis curves were obtained by pulling the sutures to a set-up load of 200 MPa and then reducing strain to zero. FIG. 6B represents normalized hysteresis curves of the SVX-PCL enriched fibers (Δ) and of MONOMAX® (∘) as a control. Each curve was normalized to the maximum strain achieved at 200 MPa, demonstrating the differences in energy loss (marked by an arrow). FIG. 6C represents a bar graph showing percentage of energy loss for SVX-PCL enriched fibers (Δ, n=5) and for MONOMAX® (∘, n=3), as calculated from the curves represented by FIG. 6A. The percentage of energy loss was calculated by dividing the area under the relaxation curve, by the area under the pulling curve. SVX-PCL enriched polymeric fibers show a significant reduction of energy loss (about 20% reduction) as compared to MONOMAX®. Data are means; p-value>0.05.

FIGS. 7A-B represent degradation profile of the enriched polymeric fibers (PCL fibers enriched with about 10% w/w of SVX-PCL), versus MONOMAX® as a control with increasing incubation times. FIG. 7A is a graph representing changes in Young's modulus of the SVX-PCL enriched fibers (Δ, n=5) and of MONOMAX® (∘, n=3) as a control, upon incubation with 5 M HCl at 37° C. FIG. 7B is a bar graph representing decay constants of Young's modulus for SVX-PCL enriched fibers (Δ, n=5) and for MONOMAX® (∘, n=3). SVX-PCL enriched polymeric fibers show a significant increase of the decay constant (about 140% increase of degradation time) as compared to MONOMAX®. Decay constants (τ), were calculated by fitting exponential decay curves to the values of Young's modulus as represented by FIG. 7A. Data are means; Δ, R²=0.83 and ∘, R²=0.92.

FIGS. 8A-B represent mechanical properties of the enriched polymeric fibers (23:1 PP:PCL fibers enriched with about 10% w/w of SVX-PCL), versus PROLENE® as a control. FIG. 8A shows comparative strain-stress curves of the SVX-PCL enriched PP fibers (Δ, n=5) and of PROLENE® (□, n=4) as a control. FIG. 8B is a bar graph showing Young's modulus of the SVX-PCL enriched PP fibers (Δ) and of PROLENE® (□) as a control. SVX-PCL enriched PP fibers show increased (about 70%) stiffness (Young's modulus) as compared to PROLENE®. Data are means; p-value>0.05.

FIGS. 9A-B represent knot strength of the enriched polymeric fibers (23:1 PP:PCL fibers enriched with about 10% w/w of SVX-PCL), versus PROLENE® as a control. FIG. 9A shows strain-stress curves of the SVX-PCL enriched PP fibers (Δ, n=4) and of PROLENE® (□, n=5) as a control. Stress development with increasing strain was measured upon formation of a knotted suture (knot was generated in accordance with USP monograph for Absorbable sutures). FIG. 9B shows a bar graph representing knot pull tensile strength (in Newton, N) of the SVX-PCL enriched PP fibers (Δ, n=4), versus PROLENE® (□, n=5) as a control. The knot strength values were calculated from the graphs of FIG. 9A. SVX-PCL enriched PP fibers exhibited knot strength comparable to the knot strength of PROLENE®. Data are means; p-value>0.05.

FIGS. 10A-C represent mechanical hysteresis of the enriched polymeric fibers (23:1 PP:PCL fibers enriched with about 10% w/w of SVX-PCL), versus PROLENE® as a control. FIG. 10A represents hysteresis curves of the SVX-PCL enriched PP fibers (Δ) and of PROLENE® (□) as a control. Hysteresis curves were obtained by pulling the sutures to a set-up load of 200 MPa and then reducing strain to zero. FIG. 10B represents normalized hysteresis curves of the SVX-PCL enriched PP fibers (Δ) and of PROLENE® (Q) as a control. Each curve was normalized to the maximum strain achieved at 200 MPa, demonstrating the differences in energy loss (marked by an arrow). FIG. 10C represents a bar graph showing percentage of energy loss for SVX-PCL enriched PP fibers (Δ, n=4) and for PROLENE® (□, n=4), as calculated from the curves represented by FIG. 10A The percentage of energy loss was calculated by dividing the area under the relaxation curve, by the area under the pulling curve. SVX-PCL enriched PP fibers show a significant reduction of energy loss (about 10% reduction) as compared to PROLENE®. Data are means; p-value>0.05.

FIG. 11 is a graph representing changes in Young's modulus of the enriched polymeric fibers (23:1 PP:PCL fibers enriched with about 10% w/w of SVX-PCL, Δ), versus PROLENE® (□) as a control with increasing incubation times. Young's modulus of the fibers was measured upon incubation with 5 M HCl at 37° C. SVX-PCL enriched PP fibers show significant strength retention for a period of 30 days.

### DETAILED DESCRIPTION OF THE INVENTION

The invention provides, in some embodiments, compositions comprising at least one major ampullate spidroin protein (MaSp)-based fiber, and a polymer bound to the MaSp-based fiber. The invention further provides articles comprising the compositions, and methods of use and preparation of same.

The present invention is based, in-part, on the surprising findings that a polymer enriched with a composition comprising a MaSp-based fiber bound to PCL or PLA exhibits improved mechanical properties (e.g., Young's modulus and tensile strength), as exemplified hereinbelow. In some embodiments, the polymer enriched with the composition is bound to the MaSp-based fiber. In some embodiments, the polymer enriched with the composition is physically bound to the MaSp-based fiber. In some embodiments, the polymer fills at least a portion of the pores on or within the MaSp-based fiber. The disclosed composition harnesses the mechanical properties of the disclosed fibers. Different materials can be enriched or produced with the disclosed compositions such as sutures and other medical devices.

According to some embodiments, the present invention provides a composition comprising a MaSp-based fiber, and a first polymer bound to the MaSp-based fiber. In some embodiments, the MaSp-based fiber comprises a plurality of pores. In some embodiments, the MaSp-based fiber is as described hereinbelow. In some embodiments, the composition comprises a plurality of MaSp-based fibers. In some embodiments, the plurality of MaSp-based fibers comprises fibers having a different chemical composition and/or a different molecular weight (MW).

In some embodiments, bound is via a non-covalent bond, a physical interaction or both. In some embodiments, bound is via a covalent bond. In some embodiments, bound is via a non-covalent bond, a covalent bond, a physical interaction or any combination thereof.

In some embodiments, the composition comprises a MaSp-based fiber, and a first polymer substantially bound to the MaSp-based fiber via a non-covalent bond, a physical interaction or both. In some embodiments, the composition comprises comprising a MaSp-based fiber, and a first polymer substantially bound to the MaSp-based fiber, wherein bound is as described hereinabove. In some embodiments, substantially comprises at least 50%, at least 60%, at least 70%, at least 75%, at least 80%, at least 85%, at least 90%, at least 95%, at least 97%, at least 99%, by weight of the first polymer. In some embodiments, substantially comprises at least 50%, at least 60%, at least 70%, at least 75%, at least 80%, at least 85%, at least 90%, at least 95%, at least 97%, at least 99%, of the first polymer is bound to the MaSp-based fiber via a non-covalent bond, via a physical interaction or both.

In some embodiments, the first polymer is substantially bound to the MaSp-based fiber via a physical interaction, wherein substantially is as described hereinabove. In some embodiments, the physical interaction is referred to polymer chains entangled or intertwined with the MaSp-based fiber, entrapped within a mesh or a matrix formed by the MaSp-based fiber. In some embodiments, the first polymer bound to the MaSp-based fiber form a network or matrix comprising intertwisted polymeric chains and MaSp-based fibers. In some embodiments, the matrix is formed by a chain of the first polymer filling at least a portion of the pores within the MaSp-based fibers.

In some embodiments, the first polymer is substantially bound or adhered to the MaSp-based fiber via a non-covalent bond. Non-covalent bonds are well-known in the art and include inter alia hydrogen bonds, p-p stacking, Van der Waals interactions, etc.

In some embodiments, the composition comprises a composite, wherein the composite comprises the first polymer bound to the MaSp-based fiber, wherein bound is via a non-covalent bond, a covalent bond, a physical interaction or any combination thereof.

In some embodiments, the composition further comprises a second polymer. In some embodiments, the second polymer has a molecular weight in the range of 1000 Da to 1000 kDa. In some embodiments, the composition comprises the first polymer bound to the MaSp-based fiber, so as to form a composite and a second polymer in contact with the composite. In some embodiments, in contact comprises bound or adhered, wherein bound is as described hereinabove. In some embodiments, the second polymer is bound or adhered to the MaSp-based fiber and/or to the first polymer. In some embodiments, the second polymer is bound to the MaSp-based fiber and/or to the first polymer via a non-covalent bond, a physical interaction or any combination thereof. In some embodiments, the pores of the MaSp-based fiber are filled with the first polymer

In some embodiments, the first polymer, the second polymer or both fill 50% to 100% of the volume of the pores. In some embodiments, the first polymer, the second polymer or both substantially fill 50% to 100% of the volume of the pores, wherein substantially is as described hereinabove. In some embodiments, the first polymer, the second polymer or both fill 55% to 100%, 60% to 100%, 55% to 100%, 70% to 100%, 75% to 100%, 80% to 100%, 85% to 100%, 90% to 100%, 95% to 100%, 50% to 99%, 50% to 98%, 50% to 97%, 50% to 95%, 50% to 90%, 70% to 90%, or 70% to 95% of the volume of the pores, including any range therebetween.

In some embodiments, the total volume of the MaSp-based fiber comprises 2% to 90% pore space. In some embodiments, the total volume of the MaSp-based fiber comprises 2% to 85%, 5% to 85%, 10% to 85%, 15% to 85%, 20% to 80%, 25% to 85%, 30% to 85%, 40% to 85%, or 50% to 85% pore space.

In some embodiments, the MaSp-based fiber comprises pores with a diameter in the range 0 nm to 30 nm, 0.3 nm to 30 nm, 0.5 nm to 30 nm, 0 nm to 28 nm, 0 nm to 25 nm, 1 nm to 30 nm, 1 nm to 25 nm, 1 nm to 23 nm, 1 nm to 20 nm, 5 nm to 25 nm, 10 nm to 30 nm, or 10 nm to 25 nm, including any range therebetween.

As used herein, the term “pore” refers to portions of a matrix that do not contain the material that makes up the solid network of the matrix; the pores are filled with any substance that is different from the substance that makes up the majority of the solid network. In some embodiments, the pores are devoid of added solid and liquid matter, although it is likely the pores contain gas.

In some embodiments, the second polymer has a molecular weight in the range of 1000 Da to 1000 kDa. In some embodiments, the second polymer has a molecular weight in the range of 1500 Da to 1000 kDa, 1000 to 2000 Da, 2000 to 3000 Da, 3000 to 5000 Da, 5000 to 7000 Da, 7000 to 10,000 Da, 10 to 20 kDa, 20 to 30 kDa, 30 to 40 kDa, 40 to 50 kDa, 50 to 60 kDa, 60 to 70 kDa, 70 to 80 kDa, 80 to 90 kDa, 90 to 100 kDa, 100 to 150 kDa, 150 to 200 kDa, 200 to 300 kDa, 300 to 400 kDa, 400 to 500 kDa, 500 to 700 kDa, 700 to 1000 kDa, 5000 Da to 1000 kDa, 10000 Da to 1000 kDa, 3000 Da to 1000 kDa, 5000 Da to 1000 kDa, or 8000 Da to 1000 kDa, including any range therebetween.

As used herein, the terms “molecular weight” or “average molecular weight” refer to a number average molecular weight (Mn) The number average molecular weight (Mn) measuring system requires counting the total number of molecules in a unit mass of polymer irrespective of their shape or size.

In some embodiments, the second polymer comprises a partially branched polymer.

As used herein, the term “branched polymer” refers to any non-linear polymer, or partially crosslinked polymer, wherein either the non-linear and/or partially crosslinked polymer may contain linear polymer portion(s), and combinations thereof. The term “branched polymer” does not refer to a 100% completely linear polymer.

In some embodiments, the first polymer has a molecular weight in the range of 100 Da to 1000 kDa. In some embodiments, the first polymer has a molecular weight in the range of 200 Da to 1000 kDa, 300 Da to 1000 kDa, 500 Da to 1000 kDa, 600 Da to 1000 kDa, 800 Da to 1000 kDa, 900 Da to 1000 kDa, 1000 Da to 1000 kDa, 1500 Da to 1000 kDa, 2000 Da to 1000 kDa, 5000 Da to 1000 kDa, 10000 Da to 1000 kDa, 3000 Da to 1000 kDa, 5000 Da to 1000 kDa, or 8000 Da to 1000 kDa, including any range therebetween.

In some embodiments, the first polymer comprises polymeric chains characterized by a molecular weight distribution between 1 and 20, between 1 and 3, between 3 and 5, between 5 and 7, between 7 and 10, between 10 and 15, between 15 and 20 including any range or value therebetween. Molecular weight distribution is well-known in the art and is calculated according to formula M̅n/\hat{M}w.

In some embodiments, the first polymer comprises a plurality of polymers, wherein the polymers are as described hereinbelow. In some embodiments, the first polymer comprises a co-polymer (e.g. a block co-polymer). In some embodiments, the block co-polymer comprises a plurality of biodegradable polymeric blocks.

In some embodiments, the term “polymer”, as used herein throughout, describes a substance, e.g., an organic substance, but alternatively an inorganic substance, composed of a plurality of repeating structural units (referred to interchangeably as backbone units or monomeric units) covalently connected to one another and forming the polymeric backbone of the polymer. The term “polymer” as used herein encompasses organic and inorganic polymers and further encompasses one or more of a homopolymer, a copolymer or a mixture thereof (e.g., a blend). The term “homopolymer” as used herein describes a polymer that is made up of one type of monomeric units and hence is composed of homogenic backbone units. The term “copolymer” as used herein describes a polymer that is made up of more than one type of monomeric units and hence is composed of heterogenic backbone units. The heterogenic backbone units can differ from one another by the pendant groups thereof.

For the sake of simplicity, the terms “polymer” and “polymeric backbone” as used herein throughout interchangeably, relate to both homopolymers, copolymers and mixtures thereof.

In some embodiments, the polymer (e.g. the first polymer, the second polymer or both) is a synthetic polymer. In some embodiments, the polymer is a thermoplastic polymer. In some embodiments, the polymer is a biodegradable polymer (e.g. a polyester, a polyether, a polyamide). In some embodiments, the polymer is a thermoset. In some embodiments, the polymer is an epoxy. In some embodiments, the polymer is polyester. In some embodiments, the polymer is selected from the group consisting of polyamides, polyurethane, Nylon, polyacrylate, polycarbonate, and silicon. In some embodiments, the polymer is a cross-linked polymer. In some embodiments, the polymer is copolymer. In some embodiments, the polymer is in the form of a hydrogel.

Non-limiting examples of biodegradable polymers comprise but are not limited to poly(lactic-co-glycolic acid), poly(lactic acid), poly(glycolic acid), poly-l-lactide (PLLA), poly-d,l-lactide (PLA), polycaprolactone, polydioxanone, polyvinylalcohol (PVA), polysaccharides, polycarbonate, polyurethane, polyhydroxyalkanoates such as polyhydroxybutyrates, polyhydroxyvalerates and/or copolymers thereof, polyhydroxyalkanoate, polyropyleneglycol, polyglycolide-co-caprolactone, polyglycolide-co-trimethylene carbonate, polyethyleneglycol (PEG), poly(tetramethylene ether)glycol, polyglycolides, polydioxanones, polyhydroxybutyrate, polyhydroxyvalerate, polyphosphoester, polyamino acid or any combination or a copolymer thereof.

In some embodiments, the first polymer and the second polymer are each independently selected from the group consisting of liquid crystal polymers, maleic anhydride grafted polypropylene, polyamides, Nylon 4,6, Nylon 6, Nylon 6,6, Nylon 11, Nylon 12, poly(arylamide), polyethylene, polybutylene terephthalate, polyethylene terephthalate, polyphenylene sulfide, polyphthalamide, polyethylene (PE), polypropylene (PP), poly(vinylidene fluoride), Poly(2-hydroxyethyl methacrylate) (pHEMA), polyurethane, polyvinyl butyral, ethylene vinyl alcohol copolymer, polylactic acid (PLA), polysilane, polysiloxane, polycaprolactone (PCL), xanthan, cellulose, collagen, elastin, keratin, cotton, rubber, cellulose, wool and any combination or mixture thereof.

In some embodiments, the first polymer is PCL and the second polymer is selected from PCL, PLA, PP, PE or any combination thereof.

In some embodiments, the first polymer is biodegradable. In some embodiments, the first polymer comprises polycaprolactone (PCL), polylactic acid (PLA) or both. In some embodiments, the first polymer is devoid of a polysaccharide. In some embodiments, the first polymer is devoid of PEG. In some embodiments, the first polymer is devoid of a protein and/or a peptide (e.g. RGD). In some embodiments, the first polymer is devoid of poly(acrylic acid) (PAA), poly(methacrylic acid) (PMAA), or poly(allylamine) (PAAm).

In some embodiments, the content of the first polymer and the second polymer within the composition is 20% to 60% (w/w). In some embodiments, the content of the first polymer and the first polymer is 25% to 60% (w/w), 30% to 60% (w/w), 35% to 60% (w/w), 40% to 60% (w/w), 20% to 55% (w/w), or 20% to 50% (w/w), including any range therebetween.

In some embodiments, the content of the first polymer and the second polymer within the composition is 60% to 95% (w/w), from 60 to 70% w/w, from 70 to 75% w/w, from 75 to 80% w/w, from 80 to 85% w/w, from 85 to 90% w/w, from 90 to 92% w/w, from 92 to 95% w/w, from 95 to 97% w/w including any range or value therebetween.

In some embodiments, the content of the second polymer within the composition is from 50 to 97% w/w, from 50 to 60% w/w, from 60 to 70% w/w, from 70 to 75% w/w, from 75 to 80% w/w, from 80 to 85% w/w, from 85 to 90% w/w, from 90 to 92% w/w, from 92 to 95% w/w, from 95 to 97% w/w including any range or value therebetween.

In some embodiments, the content of the second polymer within the composition is at least 80% w/w, at least 85% w/w, at least 87% w/w, at least 83% w/w, at least 90% w/w, at least 95% w/w, at least 97% w/w including any range or value therebetween.

In some embodiments, a composition of the invention comprises a copolymer comprising a MaSp-based fiber covalently bound to a first polymer. In some embodiments, the first polymer is as described hereinabove. In some embodiments, the copolymer is a block copolymer.

In some embodiments, the block copolymer comprises the first polymer covalently bound to a side chain and/or to the “N-terminus” of the MaSp-based fiber. In some embodiments, the block copolymer comprises a plurality of covalently bound blocks, wherein each block independently comprises the MaSp or the first polymer, wherein the first polymer is as described hereinabove. In some embodiments, the copolymer comprises first polymer covalently bound to a side chain of an amino acid and/or the “N-terminus” of the MaSp. In some embodiments, the first polymer is covalently bound to the MaSp via any of C-terminus, N-terminus, lysine, cysteine, tyrosine, histidine, aspartic acid, glutamic acid, serine and threonine or any combination thereof.

In some embodiments, the block copolymer is devoid of the first polymer bound to the MaSp via C-terminus and/or a carboxyl group of an amino acid. In some embodiments, the block copolymer is devoid of the first polymer bound to the MaSp via C-terminus, via aspartic acid, via glutamic acid or any combination thereof.

In some embodiments, the first polymer is covalently bound to the MaSp via a linker or a spacer. In some embodiments, the linker or spacer comprise a bifunctional reactive group (e.g. di-N-hydroxysuccininme (NHS) ester, di-isocyanate, di-acylhalide etc.). In some embodiments, the linker or spacer comprise at least two reactive groups interconnected by an alkyl moiety. In some embodiments, the linker or spacer comprises an electrophilic reactive group (such as NHS ester, isocyanate, acylhalide) bound to a first end of the alkyl moiety and a nucleophilic group (such as amine, thiol) bound to a second end of the alkyl moiety. Such linkers are known in the art and include inter alia hexamethylene diisocyanate, toluene diisocyanate, methylene diphenyl diisocyanate. In some embodiments, the linker or spacer comprise phosgene, or any compound related thereto (e.g. diphosgene, diphosgene, triphosgene, carbonyl diimidazole, disuccinimidyl carbonate).

In some embodiments, at least 30%, at least 40%, at least 50%, at least 60%, at least 70%, at least 80%, at least 90%, at least 95% including any range or value therebetween, of MaSp proteins within the composition are covalently bound to at least one chain of the first polymer. In some embodiments, each MaSp protein within the block copolymer is covalently bound to at most one polymer, wherein the polymer is the first polymer. In some embodiments, each MaSp protein within the block copolymer is covalently bound to at most two polymers.

In some embodiments, the copolymer is a graft copolymer. In some embodiments, the graft copolymer comprises a plurality of polymeric chains covalently bound to the MaSp-based fiber. In some embodiments, the graft copolymer comprises a plurality of polymeric chains covalently bound to MaSp protein. In some embodiments, the plurality of polymeric chains are the polymeric chains of the first polymer. In some embodiments, each MaSp protein is covalently bound to at least one chain of the first polymer. In some embodiments, each MaSp protein is grafted to at least one chain of the first polymer. In some embodiments, each MaSp protein of the graft copolymer is grafted to a plurality of chains of the first polymer. In some embodiments, the first polymer comprises one polymer. In some embodiments, the first polymer comprises a plurality of polymers having different chemical composition. In some embodiments, the first polymer is a copolymer. In some embodiments, the first polymer is a homopolymer.

In some embodiments, 1 to 100, 1 to 5, 2 to 10, 5 to 10, 10 to 20, 30 to 40, 40 to 50, 50 to 60, 60 to 80, 80 to 100 chains of the first polymer are grafted to each MaSp protein, including any range therebetween.

In some embodiments, at least 30%, at least 40%, at least 50%, at least 60%, at least 70%, at least 80%, at least 90%, at least 95% including any range or value therebetween, of MaSp proteins within the composition are grafted to at least one chain of the first polymer. In some embodiments, at least 30%, at least 40%, at least 50%, at least 60%, at least 70%, at least 80%, at least 90%, at least 95% of MaSp proteins are grafted to from 1 1 to 100, 1 to 5, 2 to 10, 5 to 10, 10 to 20, 30 to 40, 40 to 50, 50 to 60, 60 to 80, 80 to 100 chains of the first polymer including any range or value therebetween.

In some embodiments, the polymeric chains are grafted via a nucleophilic side chain of an amino acid and/or via the N-terminus of the MaSp protein. In some embodiments, the polymeric chains are grafted to the MaSp-based fiber via tyrosine, serine, lysine, histidine, cysteine, threonine or any combination thereof. In some embodiments, the polymeric chains of the graft copolymer are grafted to the MaSp protein via tyrosine, cysteine, N-terminus or a combination thereof.

In some embodiments, at least 50%, at least 60%, at least 70%, at least 80%, at least 90%, at least 95%, at least 92%, at least 85%, at least 75%, at least 98%, at least 99% of the polymeric chains of the first polymer are grafted to the MaSp-based fiber via tyrosine, cysteine or both.

In some embodiments, the graft copolymer comprises the polymeric chains of the first polymer in-situ polymerized on the MaSp-based fiber. In some embodiments, the polymeric chains of the first polymer are an in-situ polymerized via a ring-opening polymerization (ROP), as described hereinbelow.

In some embodiments, the graft copolymer of the composition is manufactured by in-situ polymerization, as described herein. In some embodiments, the graft copolymer of the composition is manufactured by the method of the invention.

In some embodiments, the copolymer (such as block copolymer and/or graft copolymer) comprises the MaSp protein modified with the first polymer. In some embodiments, the modification is substantially on the surface of the MaSp-based fiber. In some embodiments, the outer surface of the MaSp-based fiber is chemically modified, so as provide binding or adhesion properties thereto. In some embodiments, the outer surface of the MaSp-based fiber is chemically modified so as to provide hydrophobic or hydrophilic properties to the fiber. In some embodiments, the outer surface of the modified fiber has lower porosity as compared to a pristine (e.g. non-modified) fiber. In some embodiments, the outer surface of the modified fiber has lower surface roughness as compared to a pristine fiber. In some embodiments, the outer surface of the modified fiber has an enhanced surface contact angle as compared to a pristine fiber. It should be apparent, that the surface properties of the fiber can be modified by grafting different polymers thereto. For example, PCL-modified MaSp-based fiber may exhibit enhanced hydrophobicity, as compared to PEG- or PVA-modified MaSp-based fiber.

In some embodiments, the outer surface is chemically modified, so as to enhance non-covalent bonding interactions of the MaSp-based fiber. In some embodiments, the outer surface of the MaSp-based fiber is chemically modified by the first polymer. In some embodiments, the surface modified MaSp-based fiber is characterized by enhanced adhesion and/or binding to the second polymer, wherein the second polymer is as described herein. It should be apparent, that the exact choice of the first polymer may be dependent on the chemical composition of the second polymer, and/or the desired mechanical properties (such as Young's modulus, tensile strength) of the final composition.

In some embodiments, the copolymer of the invention is characterized by an increased binding or adhesion to the second polymer as compared to the composite, wherein the composite is as described hereinabove. In some embodiments, the composition comprising the copolymer bound to the second polymer is characterized by an increased mechanical strength (Young's modulus, tensile strength, elongation at break) as compared to the composite bound to the second polymer.

In some embodiments, binding or adhesion of the copolymer to the second polymer is adjustable by selecting the chemical composition of the first polymer. In some embodiments, binding or adhesion of the copolymer to the second polymer is predetermined by the chemical composition of the first polymer. In some embodiments, the chemical composition of the first polymer predetermines non-covalent interactions with the second polymer. As shown in the Examples section, a composition comprising PCL enriched with PCL-bound MaSp-based fibers exhibits superior mechanical strength as compared to a composition comprising PCL enriched with PLA-bound MaSp-based fibers.

In some embodiments, a w/w ratio of the first polymer to the MaSp-based fiber within the composition of the invention (e.g. copolymer or composite) is from 10:1 to 1:10, from 10:1 to 8:1, from 8:1 to 6:1, from 6:1 to 4:1, from 4:1 to 3:1, from 3:1 to 2:1, from 2:1 to 1:1, from 1:1 to 1:2, from 1:2 to 1:3, from 1:3 to 1:5, from 1:5 to 1:10 including any range therebetween.

In some embodiments, a w/w ratio of the first polymer to the MaSp-based fiber within the composition is at most 6:1, at most 5:1, at most 4:1, at most 3:1, at most 2:1, at most 1:1 including any range therebetween.

In some embodiments, a w/w ratio of the first polymer to the second polymer is between 1:7 and 1:20, between 1:7 and 1:15, between 1:7 and 1:10, between 1:7 and 1:8, between 1:8 and 1:10, between 1:10 and 1:15, between 1:15 and 1:20, between 1:5 and 1:10, between 1:5 and 1:7, between 1:4 and 1:5 including any range therebetween.

In some embodiments, a w/w concentration of the composite or the copolymer within the composition of the invention is between 1 and 50%, between 1 and 40%, between 1 and 30%, between 1 and 10%, between 1 and 3%, between 3 and 5%, between 5 and 7%, between 7 and 8%, between 8 and 10%, between 10 and 12%, between 12 and 15%, between 15 and 18%, between 18 and 20%, between 20 and 25%, between 25 and 30% including any range therebetween, wherein the composition comprises the composite or the copolymer of the invention and the second polymer.

In some embodiments, a w/w concentration of the composite or the copolymer within the composition is at most 30%, at most 25%, at most 20%, at most 15%, at most 10% including any range or value therebetween. Without being bound to any mechanism or theory, it is postulated that a w/w concentration of the copolymer within the composition above 30% w/w results in a material characterized by an increased fragility or brittleness and/or formation of aggregates.

In some embodiments, a homogenous composition of the invention comprises at most 30% w/w, at most 25% w/w, at most 20% w/w, at most 15% w/w of the copolymer or the composite of the invention. In some embodiments, a composition comprising at most 30% w/w, at most 25% w/w, at most 20% w/w, at most 15% w/w of the copolymer or the composite of the invention is substantially devoid of aggregates. In some embodiments, substantially is about 80%, about 90%, about 92%, about 95%, about 97%, about 98%, about 99% by weight of the composition.

In some embodiments, a composition comprising the second polymer and at most 30% w/w, at most 25% w/w, at most 20% w/w, at most 15% w/w of the copolymer or the composite of the invention is characterized by a sufficient elasticity, as expressed by a Young's modulus as described hereinbelow.

In some embodiments, a composition as described herein, further comprising a third polymer.

In some embodiments, the third polymer is selected from a synthetic polymer, a thermoplastic polymer, a thermoset an epoxy, a polyester a polyamide, a polyol, a polyurethane, polyethylene, Nylon, a polyacrylate, a polycarbonate, polylactic acid (PLA) or a copolymer thereof a silicon, a liquid crystal polymer, a maleic anhydride grafted polypropylene, polycaprolactone (PCL), rubber, cellulose, or any combination thereof.

In some embodiments, the content of the third polymer is 30% to 99% (w/w) of the total composition.

In some embodiments, the composition further comprising a surfactant. In some embodiments, the composition further comprises a silicone surfactant.

In some embodiments, the composition further comprises a carrier. In some embodiments, the composition further comprises a particle with a diameter smaller than 20 nm. In some embodiments, the composition is substantially devoid of a surfactant, a carrier, a particle or a combination thereof, wherein substantially is at least 80%, at least 90%, at least 92%, at least 95%, at least 97%, at least 98%, at least 99% by weight of the composition including any range or value therebetween.

In some embodiments, the composition further comprises 1% to 15% (w/w), 1% to 12% (w/w), 1% to 10% (w/w), 1% to 7% (w/w), 1% to 6% (w/w), or 5% to 15% (w/w) of a silicone surfactant, including any range therebetween.

In some embodiments, the composition comprises residual amounts of an organic solvent, a monomer or both, wherein the monomer is as described hereinbelow.

In some embodiments, the composition is in the form of a thread, a sheet or a film.

According to some embodiments, the present invention provides an article comprising the composition as described herein.

In some embodiments, the article comprises a biocompatible material.

In some embodiments, the article comprises a coating.

Non-limiting examples of the article include the form of a suture, surgical mesh, medical adhesive strips, electrospun mesh, skin grafts, fat grafts, cosmetics, dermal fillers replacement ligaments, drug eluting/delivery device clothing fabric, bullet-proof vest lining, cable, tube, film, rope, fishing line, tires, sports equipment and reinforced plastics.

In some embodiments, the composition of the present invention can be used or incorporated in any article for which desired characteristics are high tensile strength and elasticity.

In some embodiments, the composition or the article of the invention comprises a polymer enriched by the copolymer or by the composite, wherein the copolymer or the composite are as described hereinabove. In some embodiments, the composition of the invention comprises the second polymer enriched by the copolymer or by the composite.

In some embodiments, the article in a form a surgical suture comprises the second polymer enriched by the copolymer of the invention. In some embodiments, the second polymer comprises a biodegradable polymer (such as PCL). In some embodiments, the second polymer comprises polyolefin (such as PP). In some embodiments, the second polymer comprises a mixture of PCL and PP. In some embodiments, the second polymer comprises polyolefin and at most 30%, at most 20%, at most 15%, at most 10%, at most 8%, at most 5%, at most 4%, at most 3%, at most 2%, at most 1% of the biodegradable polymer (such as PCL).

In some embodiments, the second polymer is enriched by the copolymer, wherein the first polymer is a biodegradable polymer (such as PCL).

In some embodiments, the second polymer is enriched by at least 3% w/w, by at least 5% w/w, by at least 7% w/w, by at least 10% w/w, by at least 12% w/w, by at least 15% w/w, by at least 20% w/w, by at least 25% w/w, by at least 30% w/w including any range or value therebetween.

In some embodiments, enrichment is between 0.1% and 30%, between 0.1% and 0.5%, between 0.5% and 1%, between 1% and 3%, between 3% and 5%, between 5% and 7%, between 7% and 9%, between 9% and 12%, between 12% and 15%, between 15% and 20%, between 20% and 25%, between 25% and 30% including any range or value therebetween, by total weight of the composition or the article comprising thereof.

In some embodiments, the enriched polymer or an article comprising thereof is characterized by enhanced mechanical strength (e.g. Young's modulus, tensile strength, knot strength, mechanical hysteresis) relative to non-treated polymer, as described herein. In some embodiments, the enriched polymer or an article comprising thereof is characterized by enhanced chemical stability (e.g. upon incubation with a concentrated strong acid solution) relative to non-treated polymer, as described herein.

In some embodiments, the enriched polymer or an article comprising thereof is characterized by Young's modulus of at least 600 MPa, at least 700 MPa, at least 800 MPa, at least 900 MPa, at least 1000 MPa, at least 1100 MPa, at least 1200 MPa, at least 1500 MPa, at least 1700 MPa, at least 2000 MPa, including any range or value therebetween.

In some embodiments, 3% w/w enrichment increases the Young's modulus of the polymer by at least 15%, at least 20%, at least 25%, at least 30%, at least 40%, at least 50% including any range or value therebetween, wherein the enriched polymer is in a form of a film.

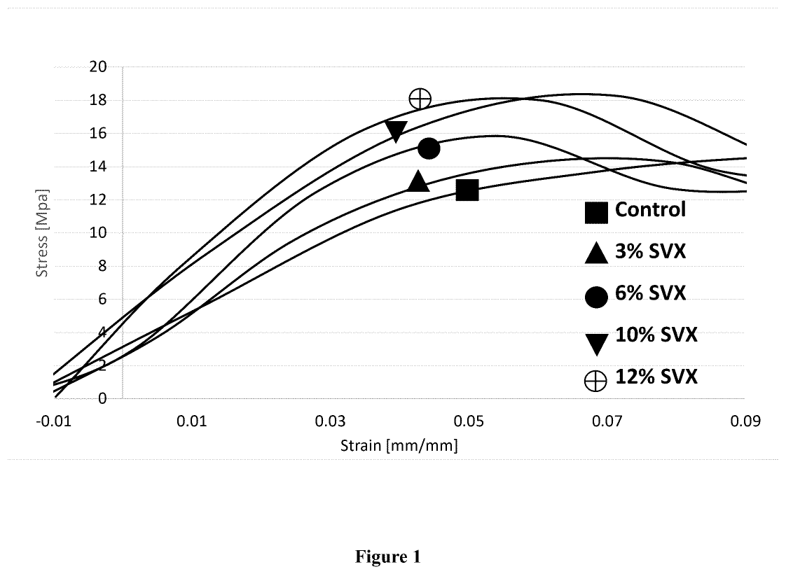

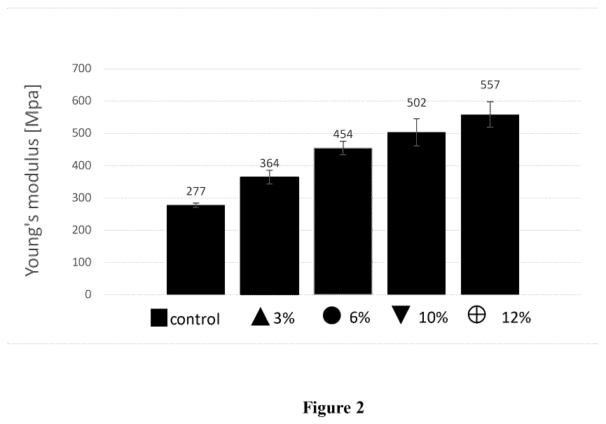

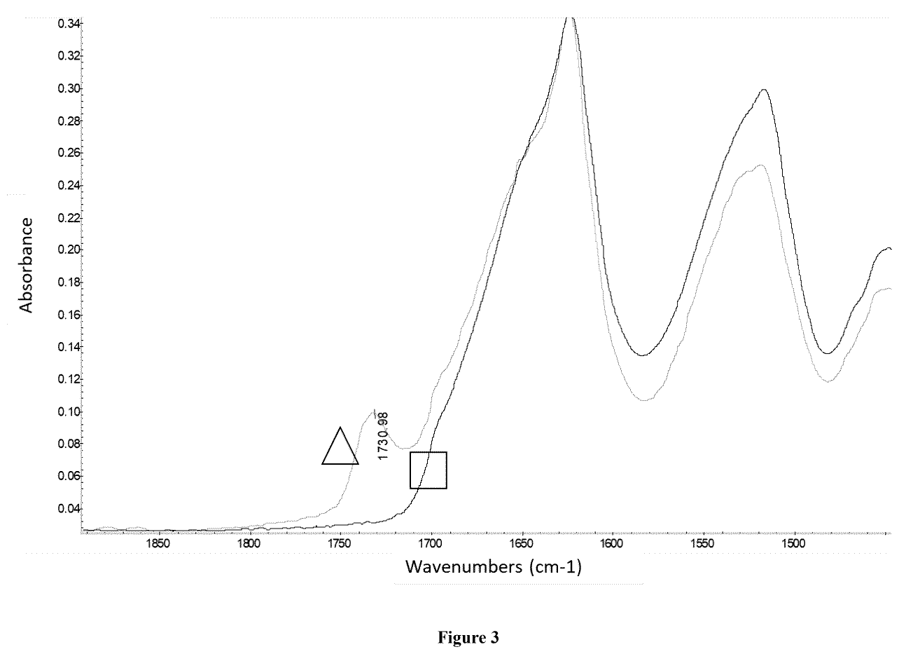

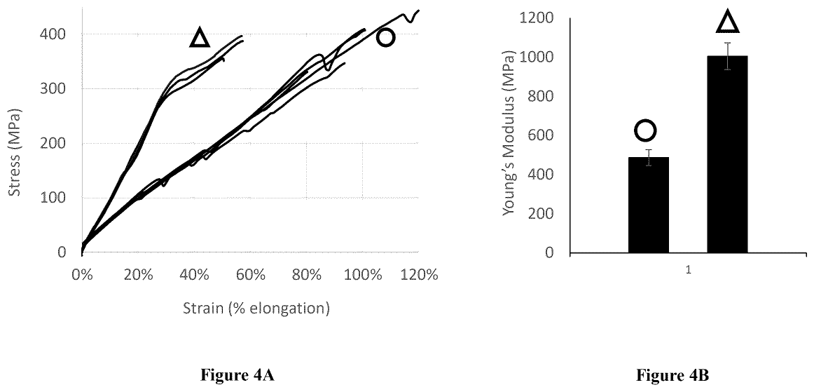

In some embodiments, 10% w/w enrichment increases the Young's modulus of the polymer by at least 30%, at least 35%, at least 40%, at least 50%, at least 60%, including any range or value therebetween wherein the enriched polymer is in a form of a film. The mechanical properties of enriched polymeric films are exemplified in the Examples section and represented by FIG. 1.

In some embodiments, mechanical strength of a fiber is sufficiently greater than the mechanical strength of a polymeric film, as represented by FIGS. 1 and 4.

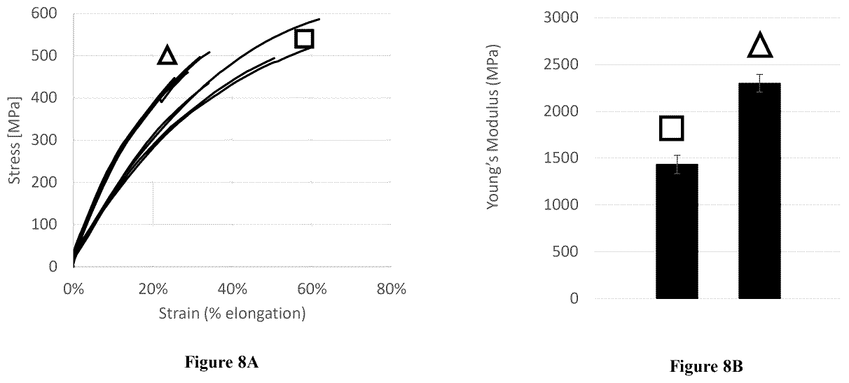

In some embodiments, 8-10% w/w enrichment increases the stiffness (Young's modulus) of the article (such as an enriched fiber) by at least 50%, at least 60%, at least 70%, at least 80%, at least 90%, at least 100%, at least 120%, including any range or value therebetween as compared to a pristine fiber (i.e. non enriched fiber), as shown by FIGS. 4A-B and by FIG. 8A-B.

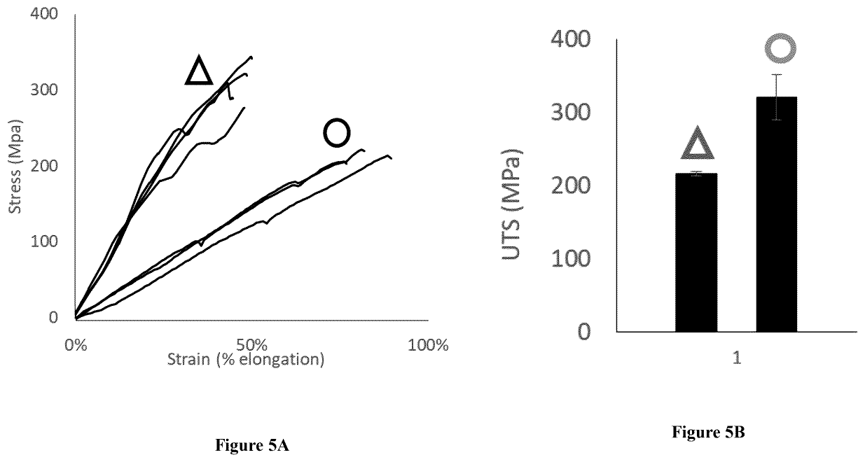

In some embodiments, 8-10% w/w enrichment increases knot strength of the enriched fiber by at least 30%, at least 40%, at least 50%, at least 60%, at least 70%, including any range or value therebetween as compared to a pristine fiber (i.e. non enriched fiber) as shown by FIGS. 5A-B. In some embodiments, about is as described herein.

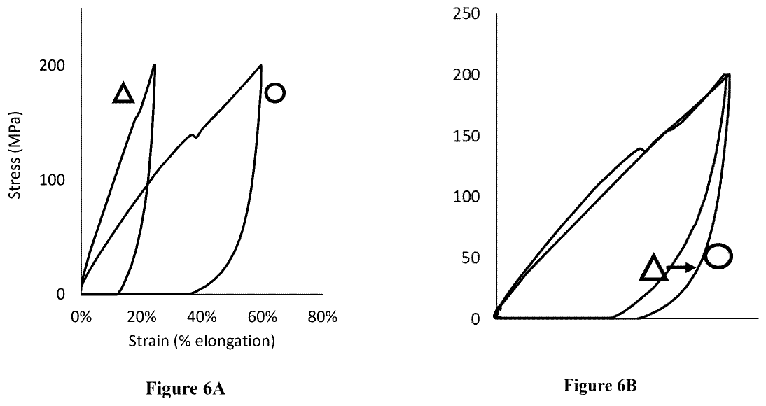

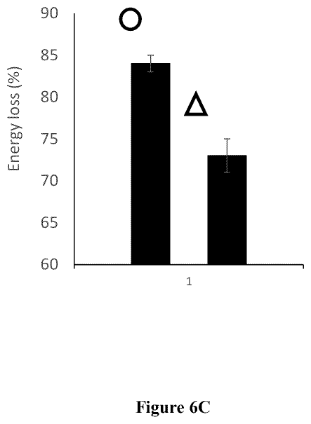

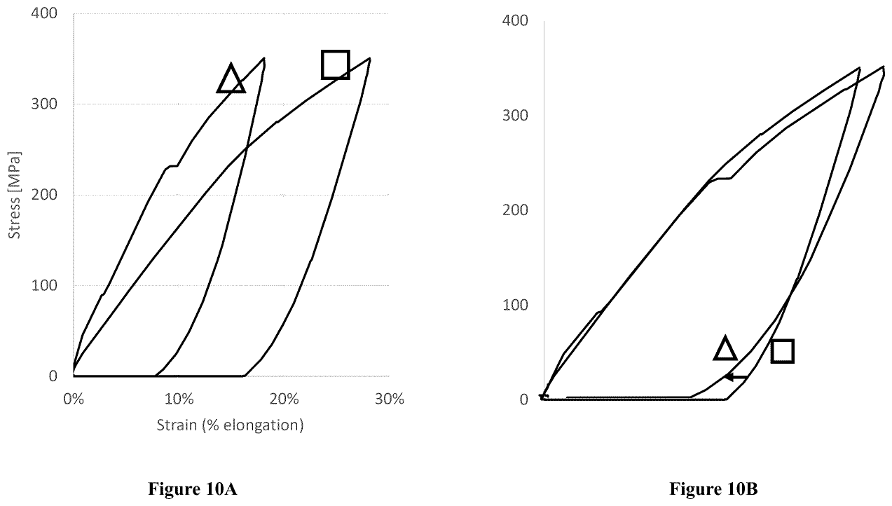

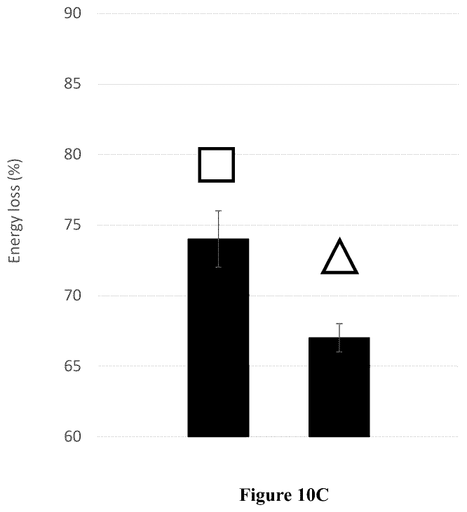

In some embodiments, 8-10% w/w enrichment increases mechanical hysteresis of the enriched fiber by at least 3%, at least 5%, at least 7%, at least 9%, at least 10%, at least 15%, at least 20%, including any range or value therebetween as compared to a pristine fiber (i.e. non enriched fiber) as shown by FIGS. 6A-C and by FIGS. 10A-C. In some embodiments, about is as described herein.

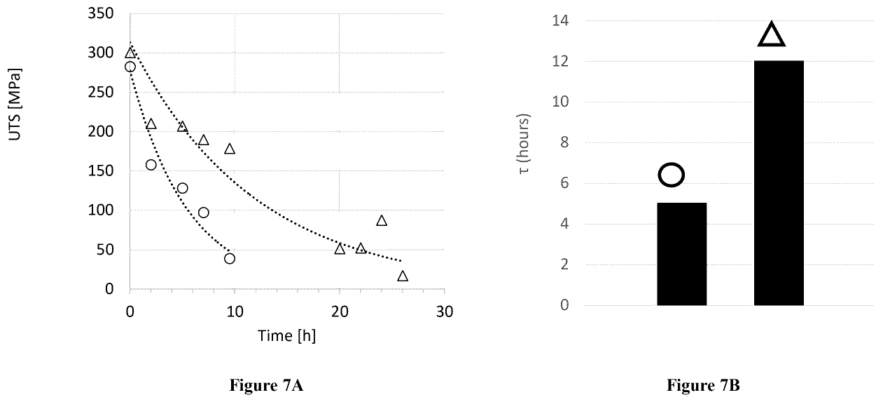

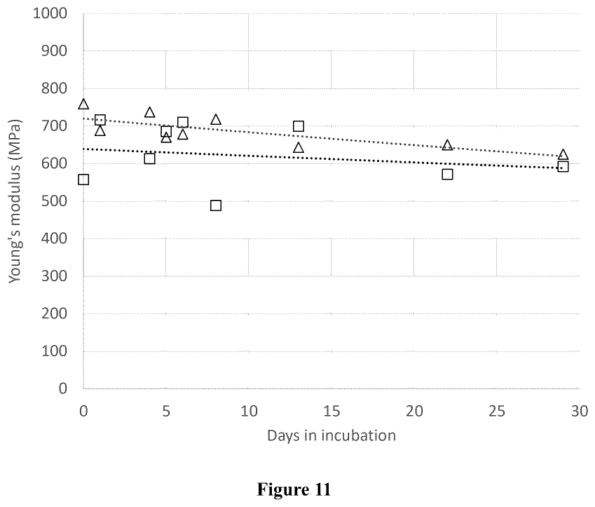

In some embodiments, 8-10% w/w enrichment increases chemical stability of the enriched fiber, as represented by FIGS. 7 and 11. In some embodiments, 8-10% w/w enrichment of PCL fibers results in about 140% increase of degradation time (FIG. 7).

In some embodiments, 8-10% w/w enriched polyolefin (such as PP or a combination of PP and PCL) fibers are substantially stable to 5M HCL at 37° C. (FIG. 11), wherein stable refers to ability of the fiber to maintain its mechanical properties. In some embodiments, at least 50%, at least 60%, at least 70%, at least 80%, at least 90% of 8-10% w/w enriched polyolefin fibers are stable to 5M HCL at 37° C. for at least 30 days.

Adaptability and use of the compositions and articles described herein in other forms, such as a dry spray coating, bead-like particles, or use in a mixture with other compositions are also contemplated by the present invention.

According to some embodiments, the present invention provides a surgical suture comprising the composition as described herein.

In some embodiments, the present invention provides a surgical suture comprising a first polymer bound at least one MaSp-based fiber, and a second polymer having a molecular weight in the range of 1000 Da to 1000 kDa, wherein bound is as described herein. In some embodiments, the pores of the MaSp-based fiber are partially filled with the first polymer, the second polymer or both. In some embodiments, the first polymer and the second polymer comprise PCL. In some embodiments, the first polymer the second polymer or both are devoid of PLA, PAA, PMAA, PAAm, PEG polysaccharide, a protein and/or a peptide (e.g. RGD).

In some embodiments, the surgical suture further comprises a third polymer. In some embodiments, the third polymer comprises PCL.

According to some embodiments, the present invention provides a medical device comprising the composition as described herein.

In some embodiments, the present invention provides a medical device comprising at least one MaSp-based nanofiber matrix, a first polymer having a molecular weight in the range of 1000 Da to 1000 kDa and a second polymer covalently bound to the nanofiber matrix. In some embodiments, the first polymer and the second polymer comprise PCL. In some embodiments, the medical device further comprises a third polymer. In some embodiments, the third polymer comprises PCL.

In some embodiments, a medical device is coated with the composition as described herein. In some embodiments, the composition is incorporated within the medical device.

### Mechanical Properties

In some embodiments, the disclosed composite is characterized by an improved mechanical property as compared to a reference material. In some embodiments, the term “reference material” refers to a same chemical composition as in the composite, being free of the one or more fibers. In some embodiments, the term “reference material” refers to a plain polymeric matrix (i.e. not comprising the fibers).

By “improved mechanical property” it is meant to refer to having a more desirable mechanical property.

In some embodiments, the improved mechanical property refers to an elastic modulus. In some embodiments, the phrase “elastic modulus” refers to Young's modulus. In some embodiments, the phrase “elastic modulus” is determined by response of a material to application of tensile stress (e.g., according to procedures known in the art).

In some embodiments, the improved mechanical property refers to Flexural modulus. As used herein and in the art, the flexural modulus (also referred to as “bending modulus”) is the ratio of stress to strain in flexural deformation, or the tendency for a material to bend. Flexural modulus may be determined from the slope of a stress-strain curve.

In some embodiments, the property is selected from, without being limited thereto, Young's modulus, tensile strength, fracture strain, yield point, toughness, abrasion resistance, stiffness, creep resistance, work-to-failure, stress and percentage of elongation. In some embodiments, tests such as abrasion tests may also be performed in accordance with DIN.

Stiffness refers to the slope of the linear portion of a load-deformation curve. Work to failure refers to the area under the load-deformation curve before failure. Each of these can be measured and calculated by methods standard known in the art.

In some embodiments, the tensile strength of a material refers the maximum amount of tensile stress that it can take before failure, for example breaking.

In some embodiments, the term “tensile strength” as used herein is the maximum amount of force as measured e.g., in Newton's that a material can bear without or prior to tearing, breaking, necking forming microcracks or fractures.

By “tearing, breaking, necking forming microcracks or fractures” it is meant to refer to a permanent deformation. In some embodiments, the term “permanent deformation” does not include microcracks or fractures. In some embodiments, by “permanent deformation” it is meant to refer to relative to at least 0.1%, 0.2%, 0.3%, 0.4%, 0.5%, or 1% of the original dimension or structure, including any value therebetween.

In some embodiments, the term “fracture strain” means the strain (displacement) at fracture, as determined e.g., by the stress-strain curve in a tensile test.

In some embodiments, the term “yield point” refers to the stress at which the stress-strain curve has a plateau and the elastic limit is reached. As used herein, “creep” is a measure of the change in tensile strain when a polymer sample is subjected to a constant tensile stress, for instance, gravity or applied mechanical or physical stress. Put differently, creep is the tendency of a solid material to slowly move or deform permanently under the influence of a constant tensile stress. As used herein, the term “creep resistance” refers to a polymer's ability to resist any kind of distortion when under a load over an extended period of time. “Improved creep resistance” may refer to improvement by e.g., 20 percent of the time to e.g., 5% tensile strain.

In some embodiments, the term “stress at elongation” refers to the force that acts on the material in the stretched condition. For example, “stress at 100% elongation” refers to the force that acts on the material stretched to twice its length.

In some embodiments, one or more properties selected from Young's modulus, tensile strength, yield point, mechanical hysteresis, and stress at elongation, is enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500% including any value therebetween.

In some embodiments, one or more properties selected from Young's modulus, tensile strength, yield point, mechanical hysteresis, and stress at elongation, is enhanced by e.g., at least 100%, at least 150%, at least 250%, at least 250%, at least 260%, at least 270%, at least 280%, at least 290%, at least 300%, at least 350%, at least 400%, at least 450%, at least 500%, at least 550%, at least 600%, at least 650%, at least 700%, at least 750%, at least 800%, at least 850%, at least 900%, at least 1000%, at least 1500%, at least 2000%, at least 2500%, or at least 3000% including any value therebetween.

In exemplary embodiments, the % enrichment of the PU (polyuerthane) is 25%, by weight, providing an improvement of 2700% of the Young's modulus.

In some embodiments, at least two properties selected from Young's modulus, tensile strength, yield point, mechanical hysteresis, and stress at elongation, is enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500% including any value therebetween.

In some embodiments, at least three properties selected from Young's modulus, tensile strength, yield point, mechanical hysteresis, and stress at elongation, are enhanced by e.g., at least 1%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500% including any value therebetween.

In some embodiments, the Young's modulus is enhanced by e.g., at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 50%, at least 100%, at least 200%, or at least 500% including any value therebetween.

In some embodiments, the tensile strength is enhanced by e.g., at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, or at least 50% including any value therebetween.

In some embodiments, the yield point is enhanced by e.g., at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, or at least 50% including any value therebetween.

In some embodiments, the composition is characterized by a structural strength or stability, wherein more than 20% of the structural strength results from the incorporated protein fiber(s). In some embodiments, the composite is characterized by a structural strength, wherein more than 30% of the structural strength results from the incorporated protein fiber(s). In some embodiments, the structural strength or stability is as described herein.

In some embodiments, the composition is characterized by a structural strength, wherein more than 1% of the tensile strength results from the incorporated fiber(s). In some embodiments, the composite is characterized by a structural strength, wherein more than 5% of the tensile strength results from the incorporated fiber(s). In some embodiments, the composite is characterized by a structural strength, wherein more than 10% of the tensile strength results from the incorporated fiber(s). In some embodiments, the composite is characterized by a structural strength, wherein more than 20% of the tensile strength results from the incorporated fiber(s). In some embodiments, the composite is characterized by a tensile strength, wherein more than 30% of the structural strength results from the incorporated fiber(s).

In some embodiments, the phrase “structural strength”, as used herein, refers to the ability of the composition to maintain its mechanical properties such as, without being limited thereto, elastic modulus, tensile stress, elongation (strain) and toughness or stiffness [e.g., combination of tensile stress and elongation (strain)].

### The Method

According to some embodiments, the present invention provides a method for producing the composition according to the present invention comprising the steps of: a. mixing at least one major ampullate spidroin protein (MaSp)-based fiber with at least one monomer under conditions suitable for the at least one monomer to polymerize. In some embodiments, the method is for forming an in-situ polymerized polymer bound to the MaSp-based fiber. In some embodiments, the method is for forming the composite or the copolymer of the invention. In some embodiments, the in-situ polymerized polymer is the first polymer, as described hereinabove. In some embodiments, the MaSp-based fiber is as described herein.

In some embodiments, the step a of the method comprises in-situ polymerizing a monomer on top of the MaSp-based fiber. In some embodiments, the method comprises in-situ polymerizing a monomer, so as to obtain the first polymer bound to the MaSp-based fiber, wherein bound is as described hereinabove. In some embodiments, the method comprises in-situ polymerizing a monomer, so as to obtain the first polymer covalently bound to the MaSp-based fiber.

In some embodiments, the step a of the method comprises graft polymerizing the first polymer and the MaSp-based fiber by means of any graft polymerization reaction (also used herein as a condensation) known in the art.

Non-limiting examples of such condensation reaction are selected from coordination polymerization, ring-opening polymerization, free radical polymerization, living polymerization and any other methodology of graft polymerization.

By “living polymerization” it is meant to refer to a form of chain growth polymerization where the ability of a growing polymer chain to terminate has been removed. Living radical polymerization is a type of living polymerization where the active polymer chain end is a free radical.

Living polymerization may be selected from living cationic polymerization, living anionic polymerization, and atom-transfer radical (ATR) polymerization.

Several methodologies of living radical polymerization are known in the art and are conceivable to be applied in the context of the present invention, including, without limitation, reversible-deactivation polymerization, catalytic chain transfer, cobalt mediated radical polymerization, iniferter polymerization, stable free radical mediated polymerization, ATR, reversible addition fragmentation chain transfer (RAFT) polymerization, iodine-transfer polymerization (ITP), selenium-centered radical-mediated polymerization, telluride-mediated polymerization (TERP), and stibine-mediated polymerization.

The term “condensation reaction”, also referred to in the art as “step-growth process”, and the like, means reaction to form a covalent bond between organic functional groups possessing a complementary reactivity relationship, e.g., electrophile-nucleophile. Typically, the process may occur by the elimination of a small molecule such as water or an alcohol. Additional information can be found in G. Odian, Principles of Polymerization, 3rd edition, 1991, John Wiley & Sons: New York, p. 108.

In some embodiments, in-situ polymerization is by a ring-opening polymerization (ROP), as described hereinbelow.

In some embodiments, the step a of the method comprises mixing the MaSp-based fiber and the monomer under conditions suitable for the monomer to polymerize. In some embodiments, the monomer comprises a plurality of monomers.

In some embodiments, the conditions suitable for polymerization of the monomer comprise a time period between 12 hours (h) and 4 days (d), between 0.5 h and 24 h, between 0.5 and 1 h, between 1 and 2 h, between 2 and 4 h, between 4 and 8 h, between 8 and 12 h, between 1 and 2 d, between 2 and 3 d, between 3 and 4 d, including any value therebetween.

In some embodiments, the conditions suitable for polymerization comprise a temperature between 2° and 200° C., between 2° and 40° C., between 4° and 60° C., between 6° and 80° C., between 8° and 100° C., between 10° and 120° C., between 12° and 150° C., between 15° and 200° C. including any value therebetween.

In some embodiments, the conditions suitable for polymerization comprise a w/w ratio of the monomer to the MaSp-based fiber being between 1; and 10:1. In some embodiments, a w/w ratio of the monomer to the MaSp-based is from 10:1 to 1:10, from 10:1 to 8:1, from 8:1 to 6:1, from 6:1 to 4:1, from 4:1 to 3:1, from 3:1 to 2:1, from 2:1 to 1:1, from 1:1 to 1:2, from 1:2 to 1:3, from 1:3 to 1:5, from 1:5 to 1:10 including any range therebetween.

In some embodiments, a w/w ratio of the monomer to the MaSp-based fiber is at most 6:1, at most 5:1, at most 4:1, at most 3:1, at most 2:1, at most 1:1 including any range therebetween.

In some embodiments, the conditions suitable for polymerization comprise a catalyst. In some embodiments, the catalyst is at a molar ratio between 0.01 to 1, between 0.01 to 1, between 0.01 to 0.05, between 0.05 to 0.1, between 0.1 to 0.3, between 0.3 to 0.5, between 0.5 to 0.7, between 0.7 to 1 relative to a total mole number of the monomer, including any range therebetween.

In some embodiments, the conditions suitable for polymerization comprise a base (e.g. a sterically hindered organic amine). In some embodiments, the base is at a molar ratio between 0.01 to 10, between 0.01 to 1, between 0.01 to 0.1, between 0.1 to 0.5, between 0.5 to 1, between 1 to 2, between 2 to 3, between 3 to 5, between 5 to 7, between 7 to 10, relative to a total mole number of the monomer, including any range therebetween. In some embodiments, the base is an inorganic base. In some embodiments, the base is soluble in the reaction solvent.

In some embodiments, the method is for chemically modifying the MaSp-based fiber. In some embodiments, the method is for forming a copolymer comprising the in-situ polymerized polymer covalently bound to an amino acid of the MaSp protein or of the MaSp-based fiber.

In some embodiments, the plurality of monomers polymerized into a first polymer is incorporated within the pores of the MaSp-based fiber. In some embodiments, the plurality of monomers polymerized into the first polymer is covalently attached or bound to the MaSp-based fiber.

In some embodiments, the pores of the treated MaSp-based fiber are partially filled with the first polymer.

In some embodiments, the first polymer fills 50% to 100% of the volume of the pores. In some embodiments, the first polymer fills 55% to 100%, 60% to 100%, 55% to 100%, 70% to 100%, 75% to 100%, 80% to 100%, 85% to 100%, 90% to 100%, 95% to 100%, 50% to 99%, 50% to 98%, 50% to 97%, 50% to 95%, 50% to 90%, 70% to 90%, or 70% to 95% of the volume of the pores, including any range therebetween.

In some embodiments, the first polymer is covalently bound to the MaSp-based fiber.

In some embodiments, the monomer comprises is at least 2, at least 3 at least 5, or at least 10, monomers. In some embodiments, the monomer comprises a plurality of monomers having different chemical composition. In some embodiments, a first monomer polymerizes into the first polymer and an additional monomer polymerizes into an additional polymer. In some embodiments, the pores of the treated MaSp-based fiber are partially filled with the first polymer, and the additional polymer is covalently bound to the MaSp-based fiber or vice versa. In some embodiments, the first polymer, the additional polymer or both are grafted to the MaSp-based fiber via an amino acid side chain, as described herein.

In some embodiments, the in-situ polymerized polymer is a copolymer (e.g. a graft copolymer, a block copolymer). In some embodiments, a polymer comprising two different monomers is covalently bound to the MaSp-based fiber.

In some embodiments, a first monomer and a second monomer polymerize into a polymer comprising the first monomer and the second monomer, incorporated in the pores of the MaSp-based fiber.

In some embodiments, ROP is initiated by a nucleophilic side chain of an amino acid. In some embodiments, the nucleophilic side chain opens a cyclic monomer by a nucleophilic addition-substitution mechanism. In some embodiments, ROP comprises an initiation step by a nucleophilic addition of the nucleophilic side chain to the cyclic monomer. In some embodiments, the initiation step results in generation of an anion, which is capable of reacting with an additional monomer, so as to form a polymeric chain (also referred to as a propagation step). In some embodiments, the polymeric chain resulting from the propagation step comprises a terminal anionic group. In some embodiments, the terminal anionic group is neutralized by reacting with an acid (also referred to as a termination step). In some embodiments, the acid comprises a protonated base. In some embodiments, the protonated base is formed upon deprotonation of the nucleophilic side chain by the base.

In some embodiments, the amino acid is as described herein. In some embodiments, the amino acid is devoid of a carboxylic group. In some embodiments, the amino acid is any of lysine, service, cysteine, threonine, histidine and tyrosine. In some embodiments, the amino acid is devoid of lysine. In some embodiments, the amino acid is tyrosine. In some embodiments, the nucleophilic side chain is the phenol group of tyrosine. In some embodiments, the phenol group of tyrosine present in the MaSp-based fiber acts as polymerization initiator.

In some embodiments, the in-situ polymerization is performed in a dry organic solvent. In some embodiments, the MaSp-based fiber is dispersed in a solution of a dry organic solvent. In some embodiments, dry organic solvent comprises xylene. Other organic solvents are well-known in the art and comprise inter alia, dichloromethane, tetrahydrofuran, ethers, esters etc. In some embodiments, dry organic solvent is devoid of a protic solvent. Such protic solvents are well-known in the art and comprise inter alia alcohols, such as methanol, ethanol etc.

In some embodiments, dry organic solvent is substantially devoid of moisture (i.e. water). In some embodiments, the water content of dry organic solvent is less than 10,000 ppm, less than 1,000 ppm, less than 100 ppm, less than 50 ppm, less than 30 ppm, less than 10 ppm, including any value therebetween.

In some embodiments, conditions suitable for the monomer to polymerize into a are basic conditions. In some embodiments, the MaSp-based fiber and the monomer are mixed in the presence of a base. In some embodiments, the base comprises triethylamine or diisopropylethylamine.

In some embodiments, the at least one MaSp-based fiber and the monomer are mixed in the presence of a catalyst. Non-limiting examples of catalysts according to the present invention include organo-methalic complexes, methalocenes, and amino-based catalysts. In some embodiments, catalysts that can be used according to the present invention include any catalyst that can facilitate a polymerization process during exposure to UV or other type of radiation. In some embodiments, the catalyst comprises a Tin (II) complex. In some embodiments, the catalyst comprises Tin (II) 2-ethylhexanoate.

In some embodiments, the at least one MaSp-based fiber and the monomer are used in a ratio of 1:0.01 to 1:100. In some embodiments, the at least one MaSp-based fiber and the monomer are used in a ratio of 1:0.01 to 1:50, 1:0.01 to 1:30, 1:0.01 to 1:20, 1:0.01 to 1:15, or 1:0.01 to 1:12.5, including any range therebetween.

In some embodiments, the monomer comprises a lactone (e.g. ε-caprolactone). In some embodiments, the monomer comprises a lactide.

In some embodiments, the method further comprises step b of mixing the first polymer bound to the MaSp-based fiber with a solution comprising an additional polymer. In some embodiments, the additional polymer is the second polymer, wherein the second polymer is as described herein. In some embodiments, the step b comprises mixing the copolymer or the composite of the invention with a solution comprising the second polymer. In some embodiments, the step b is an enrichment step. In some embodiments, the method is for enriching the second polymer. In some embodiments, enriching is as described hereinabove.

In some embodiments, mixing of step b is by solvent casting. In some embodiments, the solvent is any organic solvent.

In some embodiments, step b results in the second polymer bound to the composite or to the copolymer of the invention. In some embodiments, bound is as described hereinabove.

In some embodiments, the pores of the MaSp-based fiber are partially filled with the second polymer. In some embodiments, the second polymer fills 50% to 100% of the volume of the pores. In some embodiments, the second polymer fills 55% to 100%, 60% to 100%, 55% to 100%, 70% to 100%, 75% to 100%, 80% to 100%, 85% to 100%, 90% to 100%, 95% to 100%, 50% to 99%, 50% to 98%, 50% to 97%, 50% to 95%, 50% to 90%, 70% to 90%, or 70% to 95% of the volume of the pores, including any range therebetween.

In some embodiments, a w/w ratio of the second polymer to the composite or the copolymer of the invention is between 100:1 to 5:1, between 100:1 to 80:1, between 80:1 to 50:1, between 50:1 to 30:1, between 30:1 to 20:1, between 20:1 to 15:1, between 15:1 to 10:1, between 10:1 to 5:1 including any range therebetween.

In some embodiments, the first polymer and the second polymer are independently selected from a synthetic polymer, a thermoplastic polymer, a thermoset an epoxy, a polyester a polyamide, a polyol, a polyurethane, polyethylene, Nylon, a polyacrylate, a polycarbonate, polylactic acid (PLA) or a copolymer thereof a silicon, a liquid crystal polymer, a maleic anhydride grafted polypropylene, polycaprolactone (PCL), rubber, cellulose, or any combination thereof.

In some embodiments, the second polymer is as described hereinabove. In some embodiments, the second polymer is a copolymer. In some embodiments, the second polymer comprises a mixture of polymers. In some embodiments, the second polymer comprises a biodegradable polymer (e.g. PCL) and a polyolefin (e.g. PP). In some embodiments, the biodegradable polymer and a polyolefin are at w/w ratio

In some embodiments, the method further comprises an enrichments step with a third polymer.

In some embodiments, the enrichment step further comprises mixing the composite or copolymer of the invention, with an organic solvent and a third polymer, thereby forming a solution.

In some embodiments, any of the first polymer or the second polymer forms non-covalent bonds with the third polymer.

In some embodiments, the first and second polymer improve the surface area of the MaSp-based fibers available for bonding with the third polymer. In some embodiments, improving the surface area of the MaSp-based fibers available for bonding with the third polymer is by at least 1 fold, at least 10 fold, at least 30 fold, at least 50 fold, at least 100 fold, or at least 500 fold, when compared with MaSp-based fibers not treated with the first polymer and the second polymer.

In some embodiments, the first and second polymer improve the surface area of the MaSp-based fibers available for wetting. As used herein improve the surface area of the MaSp-based fibers available for wetting is by at least 1 fold, at least 10 fold, at least 30 fold, at least 50 fold, at least 100 fold, or at least 500 fold, when compared with MaSp-based fibers not treated with the first polymer and the second polymer.

In some embodiments, the term “bond” refers to a covalent bond. In some embodiments, the term “bond” refers to a non-covalent bond (e.g., electrostatic bond).

As used herein, the term “covalent bond” and “valence bond” refer to a chemical bond between two atoms created by the sharing of electrons, usually in pairs, by the bonded atoms.

As used herein, the term “non-covalent bond” refers to an interaction between atoms and/or molecules that does not involve the formation of a covalent bond between them.

In some embodiments, the treated fibers with the first polymer and the second polymer, and the third polymer are used in a ratio of 1:1000 to 1:2. In some embodiments, the treated fibers with the first polymer and the second polymer, and the third polymer are used in a ratio of 1:1000 to 0.1:9.9, 1:100 to 0.1:9.9, 1:100 to 1:2, 1:50 to 1:2, or 1:50 to 0.1:9.9, including any range therebetween.

In some embodiments, an organic solvent comprises a dry solvent. In some embodiments, an organic solvent comprises a non-protonated organic solvent. In some embodiments, an organic solvent comprises xylene, toluene, benzene, or any combination thereof. In some embodiments, an organic solvent comprises toluene.

In some embodiments, the enrichment step further comprises a surfactant. In some embodiments, the enrichment step further comprises a silicone surfactant. In some embodiments, the enrichment step further comprises 1% to 15% (w/w), 1% to 12% (w/w), 1% to 10% (w/w), 1% to 7% (w/w), 1% to 6% (w/w), or 5% to 15% (w/w) of a silicone surfactant, including any range therebetween.

In some embodiments, the enrichment step comprises solvent casting the solution and letting the solution evaporate, thereby forming a film.

In some embodiments, the process is done without forming a film. In some embodiments, the treated fibers are added to the PCL suspended in an organic solvent that will evaporate during the melting of the PCL pellets inside the extruder.

In some embodiments, the method further comprises the step of extruding the film. In some embodiments, the step of extruding the film affords an extruded fiber.

In some embodiments, the film is melted and converted into a continuous filament.

As used herein, the term “filament” refers to a fiber of indefinite length, ranging from microscopic length to lengths of a mile or greater.

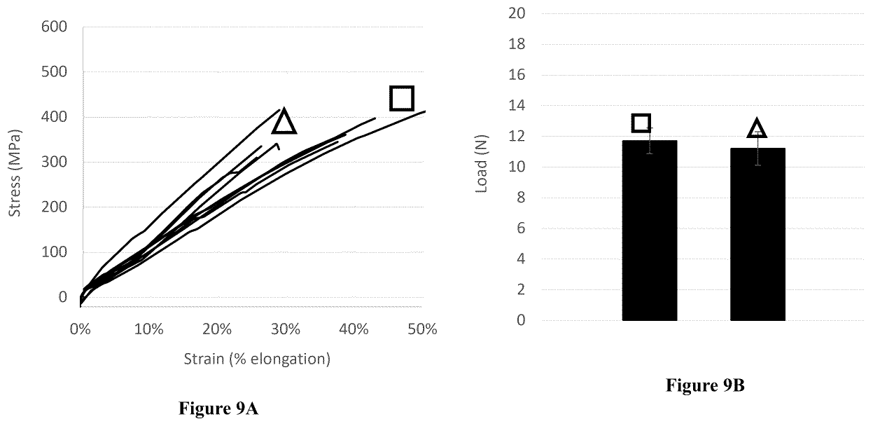

In some embodiments, the method further comprises the step of post drawing or online drawing the fiber. In some embodiments, post drawing process enhances the strength (tensile strength or Young's modulus) as represented by FIGS. 4-11. In some embodiments, drawing is by a tensioner. Other fiber drawing methods are well-known in the art.

In some embodiments, the third polymer is selected from a synthetic polymer, a thermoplastic polymer, a thermoset an epoxy, a polyester a polyamide, a polyol, a polyurethane, polyethylene, Nylon, a polyacrylate, a polycarbonate, polylactic acid (PLA) or a copolymer thereof a silicon, a liquid crystal polymer, a maleic anhydride grafted polypropylene, polycaprolactone (PCL), rubber, cellulose, or any combination thereof. In some embodiments, the third polymer is polycaprolactone (PCL).

### MaSp-Based Fiber

According to some embodiments, the present invention provides a composition comprising a MaSp-based fiber. In some embodiments, the MaSp-based fiber is present at a concentration of 0.1% to 30%, by total weight. In some embodiments, the MaSp-based fiber comprises at least one MaSp-based fiber. In some embodiments, the at least one MaSp-based fiber is present at a concentration of 0.1% to 25%, 0.1% to 20%, 0.1% to 15%, 0.5% to 30%, 1% to 30%, 5% to 30%, or 10% to 30%, by total weight, including any range therebetween.

In some embodiments, the at least one MaSp-based fiber comprises a mixture of proteins. In some embodiments, the term “SVX fiber(s)” as used herein refers to the MaSp-based fibers comprising a mixture of proteins, as described herein.

In some embodiments, each protein in the mixture comprises, independently, “n” repeats of a repetitive region of a major MaSp protein, or a functional homolog, variant, derivative or fragment thereof, wherein m and n are, independently, an integer between 2 to 70.

As used herein, the term “mixture of proteins” or “protein mixture” refers to a plurality of proteins, such as, without limitation, at least 2, at least 3, at least 4, at least 5, at least 6, at least 7, at least 8, at least 9 or at least 10 types of proteins, wherein each type of protein has a unique and uniform molecular weight.

In some embodiments, the term “mixture of proteins” or “protein mixture” refers to 20 to 40 types of proteins or 20 to 35 types of proteins. In some embodiments, the protein refers to a single folded protein.

The terms “major ampullate spidroin protein” and “spidroin protein” are used interchangeably throughout the description and encompass all known major ampullate spidroin proteins, typically abbreviated “MaSp”, or “ADF” in the case of Araneus diadematus. These major ampullate spidroin proteins are generally of two types, 1 and 2. These terms furthermore include non-natural proteins, as disclosed herein, with a high degree of identity and/or similarity to at least the repetitive region of the known major ampullate spidroin proteins. Additional suitable spider silk proteins include MaSp2, MiSp, MiSp2, AcSp, FLYS, FLAS, and flagelliform.

As used herein, the term “repetitive region”, “repetitive sequence” or “repeat” refer to a recombinant protein sequence derived from repeat units which naturally occur multiple times in spider silk amino acid sequences (e.g., in the MaSp-1 protein). One skilled in the art will appreciate that the primary structure of the spider silk proteins is considered to consist mostly of a series of small variations of a unit repeat. The unit repeats in the naturally occurring proteins are often distinct from each other. That is, there is little or no exact duplication of the unit repeats along the length of the protein. In some embodiments, the synthetic spider silks of the invention are made wherein the primary structure of the protein comprises a number of exact repetitions of a single unit repeat. In additional embodiments, synthetic spider silks of the invention comprise a number of repetitions of one unit repeat together with a number of repetitions of a second unit repeat. Such a structure would be similar to a typical block copolymer. Unit repeats of several different sequences may also be combined to provide a synthetic spider silk protein having properties suited to a particular application. The term “direct repeat” as used herein is a repeat in tandem (head-to-tail arrangement) with a similar repeat. In another embodiment, the repeat used to form the synthetic spider silk of the invention is a direct repeat. In some embodiments, the repeat is not found in nature (i.e., is not a naturally occurring amino acid sequences).

An exemplary sequence comprising repetitive sequences is ADF-4: AAAAAAASGSGGYGPENQGPSGPVAYGPGGPVSSAAAAAAAGSGPGGYGPENQ GPSGPGGYGPGGSGSSAAAAAAAASGPGGYGPGSQGPSGPGGSGGYGPGSQGPS GPGASSAAAAAAAASGPGGYGPGSQGPSGPGAYGPGGPGSSAAASGPGGYGPGS QGPSGPGGSGGYGPGSQGPSGPGGPGASAAAAAAAAASGPGGYGPGSQGPSGPG AYGPGGPGSSAAASGPGGYGPGSQGPSGPGAYGPGGPGSSAAAAAAAGSGPGGY GPGNQGPSGPGGYGPGGPGSSAAAAAAASGPGGYGPGSQGPSGPGVYGPGGPGS SAAAAAAAGSGPGGYGPGNQGPSGPGGYGPGGSGSSAAAAAAAASGPGGYGPG SQGPSGPGGSGGYGPGSQGPSGPGASSAAAAAAAASGPGGYGPGSQGPSGPGAY GPGGPGSSAAASGPGGYGPGSQGPSGPGAYGPGGPGSSAAAAAAASGPGGYGPG SQGPSGPGGSRGYGPGSQGPGGPGASAAAAAAAAASGPGGYGPGSQGPSGPGYQ GPSGPGAYGPSPSASAS (SEQ ID NO: 10). In some embodiments, the synthetic repetitive sequence of the invention is based on (e.g., has a high percentage identity, as defined hereinbelow) one or more repetitive sequences derived from ADF-4 (SEQ ID NO: 10). As used herein, the term “based on” refers to a sequence having a high percentage of homology to a repetitive sequence.

In some embodiments, each repetitive sequence comprises up to 60 amino acids, up to 55 amino acids, up to 50 amino acids, up to 49 amino acids, up to 48 amino acids, up to 47 amino acids, up to 46 amino acids, up to 45 amino acids, up to 44 amino acids, up to 43 amino acids, up to 42 amino acids, up to 41 amino acids, up to 40 amino acids, up to 39 amino acids, up to 38 amino acids, up to 37 amino acids, up to 36 amino acids or up to 35 amino acids, wherein possibility represents a separate embodiment of the present invention. In some embodiments, each repetitive sequence comprises 5 to 60 amino acids, 10 to 55 amino acids, 15 to 50 amino acids, 20 to 45 amino acids, 25 to 40 amino acids, acids, 25 to 39 amino acids or 28 to 36 amino acids, wherein possibility represents a separate embodiment of the present invention. In some embodiments, each repetitive sequence comprises 30 to 40 amino acids, 31 to 39 amino acids, 32 to 38 amino acids, 33 to 37 amino acids, 34 to 36 amino acids, wherein each possibility represents a separate embodiment of the present invention. In an additional embodiment, each repetitive sequence comprises 35 amino acids.

In some embodiments, n is an integer equal to any one of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 and 70.

In some embodiments, m is an integer equal to any one of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 and 70.

In another embodiment, the ratio of‘n’ to ‘m’ is in the range of 2:1-1:2. In another embodiment, the ratio of‘n’ to ‘m’ is in the range of 1.8:1-1:1.8. In another embodiment, the ratio of‘n’ to ‘m’ is in the range of 1.5:1-1:1.5. In another embodiment, the ratio of‘n’ to ‘m’ is in the range of 1.25:1-1:1.25. In another embodiment, the ratio of ‘n’ to ‘m’ is in the range of 1.2:1-1:1.2. The ratio of ‘n’ to ‘m’ is in the range of 1.1:1-1:1.1, in some embodiments. In another embodiment, ‘n’ and ‘m’ are equal.

In some embodiments, the n is identical for each type of protein in the mixture. The term “n is identical for each type of protein in the mixture” as used herein relates to the number of repetitive sequence for each type of protein, i.e., for one or more proteins having an identical molecular weight. As a non-limiting example, for a mixture of proteins having 16 types of proteins of differing molecular weight, each group of proteins has a different number of repetitive sequences.

In some embodiments, the various groups of proteins of the mixture have an inverse proportion between the number of repetitive sequence for each type of protein and the molar ratio of the group. In some embodiments, for each group of proteins (e.g., having an identical number of repeats), the lower the molecular weight of the proteins, the higher the molar ratio of the group.

In some embodiments, by “differing molecular weight” it is meant to refer to a molecular weights having a value that differs by at least e.g., 0.01%, 0.1%, 1%, 2%, 3%, 4%, 5%, 6%, 7%, 8%, 9%, 10%, 11%, 12%, 13%, 14%, 15%, 16%, 17%, 18%, 19%, 20%, 25%, or at least 30%.

In another embodiment, each repeat has a molecular weight in the range of 1.5 kDa to 4.5 kDa, in the range of 2 kDa to 3.5 kDa, in the range of 2.1 kDa to 3.4 kDa, in the range of 2.2 kDa to 3.3 kDa, in the range of 2.4 kDa to 3.2 kDa, in the range of 2.5 kDa to 3.1 kDa, in the range of 2.6 kDa to 3 kDa, or in the range of 2.7 kDa to 2.9 kDa, wherein each possibility represents a separate embodiment of the present invention. In another embodiment, each repeat has a molecular weight in the range of about 2.8 kDa.

In another embodiment, the composition comprises two or more proteins of the mixture having molecular weight increment of 2 kDa to 3.5 kDa, of 2.1 kDa to 3.4 kDa, of 2.2 kDa to 3.3 kDa, of 2.4 kDa to 3.2 kDa, of 2.5 kDa to 3.1 kDa, of 2.6 kDa to 3 kDa, or of 2.7 kDa to 2.9 kDa, wherein each possibility represents a separate embodiment of the present invention. In another embodiment, the composition comprises two or more proteins of the mixture having molecular weight increment of about 2.8 kDa.

In some embodiments, the repetitive region has a first moiety and a second moiety, wherein the first moiety and the second moiety are contiguous (i.e., immediately adjacent to each other). Typically, the first moiety and the second moiety are linked by a peptide bond.

In some embodiments, the first moiety of the repetitive region is an amino acid sequence of 5-30 amino acids comprising at least 95%, at least 90%, at least 85%, at least 80%, at least 75%, at least 60%, at least 55%, or at least 50% alanine residues. In some embodiments, the first moiety may comprise one or more glycine residues. In some embodiments, the first moiety comprises up to 5%, up to 10%, up to 15%, up to 20%, up to 25%, up to 30%, up to 45%, or up to 50% glycine residues.

In some embodiments, the first moiety comprises between one to fifty “n” repeats of an alanine-glycine dipeptide, such as in the formula of: (AG)₁₋₁₅. In some embodiments, the first moiety comprises between one to fifty “n” repeats of a glycine-alanine dipeptide, such as in the formula of: (GA)₁₋₁₅.

In some embodiments, the second moiety of the repetitive region is an amino acid sequence of 20-60 amino acids comprising at least 80% residues selected from the group consisting of glycine, serine, proline and tyrosine.

In some embodiments, the second moiety of the repetitive region is an amino acid sequence of 20-60 amino acids comprising at least 50%, at least 55%, at least 60%, at least 65%, at least 70%, at least 75%, at least 80%, at least 85%, at least 90%, at least 95% residues selected from the group consisting of glycine, serine, proline and tyrosine. In some embodiments, the second moiety of the repetitive region comprises not more than one or two glutamine residues. One skilled in the art will appreciate that the exact quantity and order of the glycine, serine, proline and tyrosine residues in the repetitive region may differ as long as the sequence forms self-assembling fibers.

In some embodiments, the repetitive region comprises:
(i) 10%, 11%, 12%1, 3%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% alanine residues;
(ii) 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%, 51%, 52%, 53%, 54%, 55%, 56%, 57%, 58%, 59% or 60% glycine residues;
(iii) 10%, 11%, 12%, 13%, 14%, 15%, 16%, 17%, 18%, 19%, 20%, 21%, 22%, 23%, 24%, 25%, 26%, 27%, 28%, 29% or 30% serine residues;
(iv) 10%, 11%, 12%1, 13%, 14%, 5%, 16%, 17%, 18%, 19%, 20%, 21%, 22%, 23%, 24%, 25%, 26%, 27%, 28%, 29% or 30% proline residues;
(v) 1%, 2%, 3%, 4%, 5%, 6%, 7%, 8%, 9% or 10% tyrosine residues;
(vi) 1%, 2%, 3%, 4%, 5%, 6%, 7%, 8%, 9% or 10% glutamine residues; and
(vii) 0%, 1%, 2%, 3%, 4%, 5%, arginine residues.

In some embodiments, the repetitive region comprises 13-42% of alanine residues, 25-55% glycine residues, 10-18% serine residues, 12-21% proline residues, 4-7% tyrosine residues, 4-7% glutamine residues, and 0-3% arginine residues.

In some embodiments, each of the proteins comprise, independently, an amino acid sequence as set forth in SEQ ID NO: 1

(X₁)_{Z}X₂GPGGYGPX₃X₄X₅GPX₆GX₇GGX₈GPGGPGX₉X₁₀

wherein X₁ is, independently, at each instance A or G.

In some embodiments, at least 50% of (X₁)_{Z} is A, Z is an integer between 5 to 30; X₂ is S or G; X₃ is G or E; X₄ is G, S or N; X₅ is Q or Y; X₆ is G or S; X₇ is P or R; X₈ is Y or Q; X₉ is G or S; and X₁₀ is S or G.

In some embodiments, the mixture of proteins is characterized by one or more properties selected from the group consisting of:
(a) each repeat has a molecular weight in the range of 2 kDa to 3.5 kDa, and
(b) the ratio of‘n’ to ‘m’ is in the range of 2:1 to 1:2.

In some embodiments, the n is identical for each type of protein in the mixture.

In another embodiment, n is an integer equal to or between 4 and 32. In another embodiment, m is an integer equal to or between 4 and 32. In another embodiment, the ratio of‘n’ to ‘m’ is in the range of 1.8:1-1:1.8. In another embodiment, ‘n’ and ‘m’ are equal.

In some embodiments, Z is an integer between 6 to 11, an integer between 6 to 10 or an integer between 7 to 9. In one embodiment, Z is an integer selected from 5, 6, 7, 8, 9, 10, 11, and 12. In another embodiment, Z is 8.

In another embodiment, the repetitive region of a MaSP1 protein comprises the amino acid sequence as set forth in SEQ ID NO: 2 (SGPGGYGPGSQGPSGPGGYGPGGPGSS). In another embodiment, the repetitive region of a MaSP1 protein comprises the amino acid sequence as set forth in SEQ ID NO: 3 (AAAAAAAASGPGGYGPGSQGPSGPGGYGPGGPGSS).

In another embodiment, the homolog shares at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% homology with SEQ ID NO: 1.

In another embodiment, the homolog shares at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% homology with SEQ ID NO: 2.

In another embodiment, the homolog shares at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% homology with SEQ ID NO: 3.

In another embodiment, the repetitive region of a MaSP1 protein comprises the amino acid sequence as set forth in SEQ ID NO: 4. In another embodiment, the repetitive region of a MaSP1 protein has the amino acid sequence as set forth in SEQ ID NO: 10.

In another embodiment, each protein of the mixture further comprises a single N-terminal region selected from the group consisting of: SEQ ID NO: 5 (MSYYHHHHHDYDIPTTENLYFQGAMDPEFKGLRRRAQLV); SEQ ID NO: 6 (MSYYHHHHHDYDIPTTENLYFQGAMDPEFKGLRRRAQLVRPLSNLDNAP); SEQ ID NO: 7 (MSYYHHHHHDYDIPTTENLYFQGAMDPEFKGLRRRAQLVDPPGCRNSARAGS S), or any functional homolog, variant, derivative, or fragment thereof. In another embodiment, the homolog of the C-terminal region shares at least 70% homology with any one of SEQ ID NOs: 5-7.

In another embodiment, each protein of the mixture further comprises a single C-terminal region selected from the group consisting of: SEQ ID NO: 8 (VAASRLSSPAASSRVSSAVSSLVSSGPTNGAAVSGALNSLVSQISASNPGLSGCD ALVQALLELVSALVAILSSASIGQVNVSSVSQSTQMISQALS); and SEQ ID NO:9 (GPSGPGAYGPSPSASASVAASRLSSPAASSRVSSAVSSLVSSGPTNGAAVSGALN SLVSQISASNPGLSGCDALVQALLELVSALVAILSSASIGQVNVSSVSQSTQMISQA LS), or any functional homolog, variant, derivative, fragment or mutant thereof. In another embodiment, the homolog of the N-terminal region shares at least 70% homology with SEQ ID NO: 8-9.

In some embodiments, one or more proteins of the mixture further comprises at least one tag sequence. Non-limiting examples of tags which may be used in the present invention include a His tag, a HA tag, a T7 tag, and the like. An exemplary His tag comprises six His residues or consists of six His residues as set forth in SEQ ID NO: 11 (THHHHH). In another embodiment, the tag is a HA tag comprising or consisting of the amino acid sequence as set forth in SEQ ID NO: 12 (YPYDVPDYA). In another embodiment, the tag is a T7 tag comprising or consisting of the amino acid sequence as set forth in SEQ ID NO: 13 (MASMTGGQQMG). The skilled person is well aware of alternative suitable tags or other fusion partners.

“Amino acid” as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-TUB Biochemical Nomenclature Commission.

“Amino acid sequence” or “peptide sequence” is the order in which amino acid residues, connected by peptide bonds, lie in the chain in peptides and proteins. The sequence is generally reported from the N-terminal end containing free amino group to the C-terminal end containing free carboxyl group Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, sulfhydryl bond formation, cleavage and the likes.

As used herein, “isolated” or “substantially purified”, in the context of synthetic spider silk amino-acid sequences or nucleic acid molecules encoding the same, as exemplified by the invention, means the amino-acid sequences or polynucleotides have been removed from their natural milieu or have been altered from their natural state. As such “isolated” does not necessarily reflect the extent to which the amino-acid sequences or nucleic acid molecules have been purified. However, it will be understood that such molecules that have been purified to some degree are “isolated”. If the molecules do not exist in a natural milieu, i.e. it does not exist in nature, the molecule is “isolated” regardless of where it is present. By way of example, amino-acid sequences or polynucleotides that do not naturally exist in humans are “isolated” even when they are present in humans.

The term “isolated” or “substantially purified”, when applied to an amino acid sequence or nucleic acid, denotes that the amino acid sequence or nucleic acid is essentially free of other cellular components with which they are associated in the natural state. It may be in a homogeneous state, or alternatively in either a dry or aqueous solution. Purity and homogeneity are typically determined using analytical chemistry techniques such as polyacrylamide gel electrophoresis or high-performance liquid chromatography. An amino acid sequence or nucleic acid which is the predominant species present in a preparation is substantially purified.

In some embodiments, the repeats are of a homolog, variant, derivative of a repetitive region of a MaSp1 protein or fragment thereof. In some embodiments, the repeats are of a homolog, variant, derivative of a repetitive region of an ADF-4 protein or fragment thereof.

As used herein, the term “functional” as in “functional homolog, variant, derivative or fragment”, refers to an amino acid sequence which possesses biological function or activity that is identified through a defined functional assay. More specifically, the defined functional assay is the formation of self-assembling fibers in cells expressing the functional homolog, variant, derivative or fragment.

An amino acid sequence or a nucleic acid sequence is the to be a homolog of a corresponding amino acid sequence or a nucleic acid, when the homology is determined to be at least 50%, at least 55%, at least 60%, at least 65%, at least 70%, at least 75%, at least 80%, at least 85%, at least 90%, at least 92%, at least 94%, at least 96%, at least 98% or at least 99%.

Homology, as used herein, may be determined on the basis of percentage identity between two amino acid (peptide) or DNA sequences. In general, the two sequences to be compared are aligned to give a maximum correlation between the sequences. The alignment of the two sequences is examined and the number of positions giving an exact amino acid (or nucleotide) correspondence between the two sequences determined, divided by the total length of the alignment multiplied by 100 to give a percentage identity figure. This percentage identity figure may be determined over the whole length of the sequences to be compared, which is particularly suitable for sequences of the same or very similar lengths and which are highly homologous, or over shorter defined lengths, which is more suitable for sequences of unequal length or which have a lower level of homology. Methods for comparing the identity of two or more sequences are well known in the art. Thus, for instance, programs available in the Wisconsin Sequence Analysis Package, version 9.1, for example the programs GAP and BESTFIT, may be used to determine the percentage identity between two amino acid sequences and the percentage identity between two polynucleotides sequences. BESTFIT uses the “local homology” algorithm of Smith and Waterman and finds the best single region of similarity between two sequences. BESTFIT is more suited to comparing two polypeptide or two polynucleotide sequences which are dissimilar in length, the program assuming that the shorter sequence represents a portion of the longer. In comparison, GAP aligns two sequences finding a “maximum similarity” according to the algorithm of Needleman and Wunsch. GAP is more suited to comparing sequences which are approximately the same length and an alignment is expected over the entire length. Preferably the parameters “Gap Weight” and “Length Weight” used in each program are 50 and 3 for polynucleotide sequences and 12 and 4 for polypeptide sequences, respectively. Preferably, percentage identities and similarities are determined when the two sequences being compared are optimally aligned.

The terms “identical”, “substantial identity”, “substantial homology” or percent “identity”, in the context of two or more amino acids or nucleic acids sequences, refer to two or more sequences or subsequences that are the same or have a specified percentage of amino acid residues or nucleotides that are the same (i.e., about 60% identity, or at least 65%, at least 70%, at least 75%, at least 80%, at least 85%, at least 90%, at least 91%, at least 92%, at least 93%, at least 94%, at least 95%, at least 96%, at least 97%, at least 98%, or 99% identity over a specified region (e.g., amino acid sequence SEQ ID NO: 2 or 3), when compared and aligned for maximum correspondence over a comparison window or designated region) as measured using a BLAST or BLAST 2.0 sequence comparison algorithms with default parameters described below, or by manual alignment and visual inspection. Such sequences are then to be “substantially identical”. This definition also refers to, or may be applied to, the compliment of a test sequence. The definition also includes sequences that have deletions and/or additions, as well as those that have substitutions. The preferred algorithms can account for gaps and the like.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

It should be appreciated that the invention further encompasses amino acid sequence comprising “n” repeats of a variant of any one of SEQ ID NO: 1, 2, or 3. As used herein, the term “variant” or “substantially similar” comprises sequences of amino acids or nucleotides different from the specifically identified sequences, in which one or more (e.g., 1, 2, 3, 4, 5, 6, 7, 8, 9, 10, 20 or 25) amino acid residues or nucleotides are deleted, substituted or added. The variants may be allelic variants occurring naturally or variants of non-natural origin. The variant or substantially similar sequences refer to fragments of amino acid sequences or nucleic acids that may be characterized by the percentage of the identity of their amino acid or nucleotide sequences with the amino acid or nucleotide sequences described herein, as determined by common algorithms used in the state-of-the-art. The preferred fragments of amino acids or nucleic acids are those having a sequence of amino acids or nucleotides with at least around 40 or 45% of sequence identity, preferentially around 50% or 55% of sequence identity, more preferentially around 60% or 65% of sequence identity, more preferentially around 70% or 75% of sequence identity, more preferentially around 80% or 85% of sequence identity, yet more preferentially around 90%, 91%, 92%, 93%, 94%, 95%, 96%, 97%, 98% or 99% of sequence identity when compared to the sequence of reference.

In one embodiment, a mixture of proteins is a fiber. In one embodiment, a fiber comprises a mixture of proteins. In one embodiment, a fiber comprises “m” types of proteins. In one embodiment, “m” types of proteins is a fiber. In one embodiment, “m” types of proteins is a mixture of proteins. In one embodiment, a fiber or a mixture of proteins comprises “m” types of proteins of differing molecular weight, wherein each protein in the “m” types of proteins comprises, independently, “n” repeats of a repetitive region of a major ampullate spidroin (MaSp) protein, or a functional homolog, variant, derivative or fragment thereof. In one embodiment, a mixture of proteins comprises “m” types of proteins of differing molecular weight, wherein each protein in the mixture of proteins comprises, independently, “n” repeats of a repetitive region of a major ampullate spidroin (MaSp) protein, or a functional homolog, variant, derivative or fragment thereof.

In one embodiment, a mixture of proteins or a fiber is composed of monomers. In one embodiment, a plurality of monomers are arranged in a nanofibril. In one embodiment, a plurality of nanofibrils are arranged in a fiber or make-up a fiber. In one embodiment, a monomer or a nanofibril within a mixture of proteins or a fiber has a diameter of 4 to 16 nm. In one embodiment, a monomer or a nanofibril within a mixture of proteins or a fiber has a diameter of 6 to 14 nm. In one embodiment, a monomer or a nanofibril within a mixture of proteins or a fiber has a diameter of 8 to 12 nm. In one embodiment, a fiber or a mixture of proteins has a diameter of 70 to 450 nm. In one embodiment, a fiber or a mixture of proteins has a diameter of 80 to 350 nm. In one embodiment, a fiber or a mixture of proteins has a diameter of 80 to 300 nm. In one embodiment, a fiber or a mixture of proteins has a diameter of 150 to 250 nm. In one embodiment, a fiber or a mixture of proteins is arranged as a coil. In one embodiment, a single fiber or one mixture of proteins is arranged as a coil. In one embodiment, a coil has a diameter of 5 to 800 micrometers. In one embodiment, a coil has a diameter of 5 to 500 micrometers. In one embodiment, a coil has a diameter of 5 to 30 micrometers. In one embodiment, a coil has a diameter of 5 to 20 micrometers. In one embodiment, a fiber or a mixture of proteins has a length of 5 to 800 micrometers. In one embodiment, a fiber or a mixture of proteins has a length of 30 to 300 micrometers.

In one embodiment, a composite and/or a composition as described herein comprises an amount of less than 5% or 3% fibers equal to or shorter than 5 micrometers (in length) from the total number of fibers within the composite and/or a composition. In one embodiment, a composite and/or a composition as described herein comprises an amount of less than 5% or 3% fibers equal to or shorter than 8 micrometers (in length) from the total number of the total content of fibers within the composite and/or a composition.

In one embodiment, a composite and/or a composition as described herein comprises less than 5% or 3% w/w fibers equal to or shorter than 5 micrometers (in length) from the total weight of fibers within the composite and/or a composition. In one embodiment, a composite and/or a composition as described herein comprises an amount of less than 5% or 3% w/w fibers equal to or shorter than 8 micrometers (in length) from the total weight of the total content of fibers within the composite and/or a composition.

In one embodiment, fibers equal to or shorter than 5 or 8 micrometers cause instability. In one embodiment, fibers equal to or shorter than 5 or 8 micrometers reduce the integrity of a composition or a composite as described herein. In one embodiment, fibers equal to or shorter than 5 or 8 micrometers reduce the physical strength of a composition or a composite as described herein.

In one embodiment, a fiber or a mixture of proteins is branched. In one embodiment, a fiber or a mixture of proteins comprises 1 to 10 branches. In one embodiment, a fiber or a mixture of proteins is free of carbohydrates. In one embodiment, a fiber or a mixture of proteins is non-glycosylated. In one embodiment, a fiber or a mixture of proteins is free of fat or fatty acids. In one embodiment, a fiber or a mixture of proteins is free of phosphorus. In one embodiment, “free of” is “devoid of” or essentially “devoid of”.

In one embodiment, at least 50% of proteins within a fiber or a mixture of proteins are bigger/larger/heavier (in kDa) than the median weight of the proteins within a fiber or a mixture of proteins. In one embodiment, at least 55% of proteins within a fiber or a mixture of proteins are bigger/larger/heavier (in kDa) than the median weight of the proteins within a fiber or a mixture of proteins. In one embodiment, at least 60% of proteins within a fiber or a mixture of proteins are bigger/larger/heavier (in kDa) than the median weight of the proteins within a fiber or a mixture of proteins. In one embodiment, at least 65% of proteins within a fiber or a mixture of proteins are bigger/larger/heavier (in kDa) than the median weight of the proteins within a fiber or a mixture of proteins. In one embodiment, at least 70% of proteins within a fiber or a mixture of proteins are bigger/larger/heavier (in kDa) than the median weight of the proteins within a fiber or a mixture of proteins. In one embodiment, at least 75% of proteins within a fiber or a mixture of proteins are bigger/larger/heavier (in kDa) than the median weight of the proteins within a fiber or a mixture of proteins.

In one embodiment, the aspect ratio of length to diameter of a fiber or a mixture of proteins is at least 1:10. In one embodiment, the aspect ratio of length to diameter of a fiber or a mixture of proteins is at least 1:10 to 1:1500. In one embodiment, the aspect ratio of length to diameter of a fiber or a mixture of proteins is at least 1:50 to 1:1000. In one embodiment, the aspect ratio of length to diameter of a fiber or a mixture of proteins is at least 1:100 to 1:1200. In one embodiment, the aspect ratio of length to diameter of a fiber or a mixture of proteins is at least 1:100 to 1:1000. In one embodiment, the aspect ratio of length to diameter of a fiber or a mixture of proteins is at least 1:500 to 1:1000.

The terms derivatives and functional derivatives as used herein mean the amino acid sequence of the invention with any insertions, deletions, substitutions and modifications.

It should be appreciated that by the term “insertions”, as used herein it is meant any addition of amino acid residues to the sequence of the invention, of between 1 to 50 amino acid residues, specifically, between 20 to 1 amino acid residues, and more specifically, between 1 to 10 amino acid residues. Most specifically, 1, 2, 3, 4, 5, 6, 7, 8, 9 and 10 amino acid residues. Further, the amino acid sequence of the invention may be extended at the N-terminus and/or C-terminus thereof with various identical or different amino acid residues.

Amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

In another embodiment, the repeat sequence of the invention has 17 or fewer, 16 or fewer, 15 or fewer, 14 or fewer, 13 or fewer, 12 or fewer, 11 or fewer, 10 or fewer, 9 or fewer, 8 or fewer, or 7 or fewer amino acid substitutions to the sequence of any one of SEQ ID NO: 2 or 3. In one embodiment, the repeat sequence of the invention has at least 2, at least 3, at least 4, at least 5, at least 6, at least 7, at least 8, at least 9, at least 10, at least 11, at least 12, or at least 13 amino acid substitutions to the sequence of any one of SEQ ID NO: 2 or 3.

With respect to amino acid sequences, one of skill will recognize that individual substitutions, deletions or additions to an amino acid, nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

Conservative nucleic acid substitutions are nucleic acid substitutions resulting in conservative amino acid substitutions as defined above.

Variants of the amino acid sequences of the invention may have at least 80% sequence similarity, at least 85% sequence similarity, 90% sequence similarity, or at least 95%, 96%, 97%, 98%, or 99% sequence similarity at the amino acid level, with a repeating unit denoted by any one of SEQ ID NO: 2 or 3.

The amino acid sequence of the invention may comprise “n” repeats of SEQ ID NO. 1 or SEQ ID NO. 3 or of any fragment thereof. A “fragment” constitutes a fraction of the amino acid or DNA sequence of a particular region. A fragment of the peptide sequence is at least one amino acid shorter than the particular region, and a fragment of a DNA sequence is at least one base-pair shorter than the particular region. The fragment may be truncated at the C-terminal or N-terminal sides, or both. An amino acid fragment may comprise at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 24, at least 26, at least 27, at least 28, at least 29, at least 30, at least 31, at least 32, at least 33 or at least 34 amino acids of SEQ ID NO: 1 or 3.

Mutants of the amino acid sequences of the invention are characterized in the exchange of one (point mutant) or more, about up to 10, of its amino acids against one or more of another amino acid. They are the consequence of the corresponding mutations at the DNA level leading to different codons.

Still further, the invention concerns derivatives of the amino acid sequence of the invention. Derivatives of the amino acid sequences of the invention are, for example, where functional groups, such as amino, hydroxyl, mercapto or carboxyl groups, are derivatized, e.g. glycosylated, acylated, amidated or esterified, respectively. In glycosylated derivatives an oligosaccharide is usually linked to asparagine, serine, threonine and/or lysine. Acylated derivatives are especially acylated by a naturally occurring organic or inorganic acid, e.g. acetic acid, phosphoric acid or sulphuric acid, which usually takes place at the N-terminal amino group, or at hydroxy groups, especially of tyrosine or serine, respectively. Esters are those of naturally occurring alcohols, e.g. methanol or ethanol. Further derivatives are salts, especially pharmaceutically acceptable salts, for example metal salts, such as alkali metal and alkaline earth metal salts, e.g. sodium, potassium, magnesium, calcium or zinc salts, or ammonium salts formed with ammonia or a suitable organic amine, such as a lower alkylamine, e.g. triethylamine, hydroxy-lower alkylamine, e.g. 2-hydroxyethylamine, and the like.

According to some aspects, the invention concerns an isolated nucleic acid sequence encoding two or more proteins of the mixture of proteins of the present invention. According to some embodiments, the invention provides an isolated nucleic acid sequence encoding the protein mixture of the present invention.

“Nucleic acid” refers to a molecule which can be single stranded or double stranded, composed of monomers (nucleotides) containing a sugar, phosphate and either a purine or pyrimidine. In bacteria, lower eukaryotes, and in higher animals and plants, “deoxyribonucleic acid” (DNA) refers to the genetic material while “ribonucleic acid” (RNA) is involved in the translation of the information from DNA into proteins.

Due to the degenerative nature of the genetic code it is clear that a plurality of different nucleic acid sequences can be used to code for the amino acid sequences of the invention. It should be appreciated that the codons comprised in the nucleic acid sequence of the invention may be optimized for expression in Sf9 host cells.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Within the context of the present invention, genes and DNA coding regions are codon-optimized for optimal expression in host cells, and in a specific example, Sf9 Spodopterafrugiperda insect cells.

The term “expression” as used herein is intended to mean the transcription and translation to gene product from a gene coding for the sequence of the gene product. In the expression, a DNA chain coding for the sequence of gene product is first transcribed to a complementary RNA which is often a messenger RNA and, then, the thus transcribed messenger RNA is translated into the above-mentioned gene product if the gene product is a protein.

In some embodiments, the invention relates to one or more expression vectors comprising a nucleic acid sequence encoding the proteins mixture of the invention. In some embodiments, the invention relates to one or more expression vectors comprising a nucleic acid sequence encoding at least a portion of the protein mixture of the invention (e.g., two or more group of proteins having a differing molecular weight). The amino acid sequence encoded by the nucleic acid sequence comprised within the expression vector of the invention may optionally further comprise at least one of a C-terminal region (e.g., denoted as SEQ ID NO: 8 or 9); and an N-terminal region (e.g., selected from SEQ ID NO: 5-7). It should be noted that the nucleic acid sequence is under expression control of operably linked promoter and, optionally, regulatory sequences.

As used herein, a “vector”, “expression vector” or “plasmid” as referred to herein is an extra-chromosomal element often carrying genes which are not part of the central metabolism of the cell, and usually in the form of circular double-stranded DNA molecules. It may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification and selection of cells which have been transformed or transfected with the vector. As used herein, “transformation” or “transfection” is the acquisition of new genes in a cell by the incorporation of nucleic acid. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined, namely, the expression of the synthetic spider silk proteins.

In specific embodiments, the vector is a viral vector, most specifically a baculovirus vector system or a vaccinia virus vector system. Examples of such commercially available baculovirus systems Baculo-Gold®, Flash-Bac® and the bac to bac system. Further viral vector systems may also be used in this invention. From case to case, a modification of the vector may be needed. Examples for further viral vectors are adenoviruses and all negative-strand RNA-viruses, e.g. rabies, measles, RSV, etc.

In one embodiment, a baculovirus system as used for expressing the synthetic silk protein of the invention. Baculoviruses are a family of large rod-shaped viruses that can be divided to two genera: nucleopolyhedroviruses and granulo-viruses. They have a restricted range of hosts that they can infect that is typically restricted to a limited number of closely related insect species. Because baculoviruses are not harmful to humans they are a safe option for use in research and commercial or industrial applications. Baculovirus expression in insect cells represents a robust method for producing recombinant glycoproteins, a significant advantage over prokaryotic expression which is lacking in terms of glycosylation, and consequently, proper protein folding.

As indicated above, the expression vector of the invention is operably linked to a promoter. The terms “promoter” and “promoter region” refer to a sequence of DNA, usually upstream of (5′ to) the protein coding sequence of a structural gene, which controls the expression of the coding region by providing the recognition for RNA polymerase and/or other factors required for transcription to start at the correct site. Promoter sequences are necessary but not always sufficient to drive the expression of the gene. The-term “suitable promoter” will refer to any eukaryotic or prokaryotic promoter capable of driving the expression of a synthetic spider silk variant gene.

Promoters which are useful to drive expression of heterologous DNA fragments in Sf9 are numerous and familiar to those skilled in the art. Virtually any promoter capable of driving the gene encoding a silk variant protein is suitable for the present invention. For example, polyhedrin, basic protein, p10, OpIE2 and gp4 promoters may be suitable promoters for the expression.

A coding sequence and regulatory sequences are the to be “operably linked” or “operably joined” when they are covalently linked in such a way as to place the expression or transcription of the coding sequence under the influence or control of the regulatory sequences. If the regulatory sequence is positioned relative to the gene such that the regulatory sequence is able to exert a measurable effect on the amount of gene product produced, then the regulatory sequence is operably linked to the gene. If it is desired that the coding sequences be translated into a functional protein, two DNA sequences are the to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribing and 5′ non-translating sequences involved with initiation of transcription and translation respectively, such as a TATA box, capping sequence, CAAT sequence. Especially, such 5′ non-transcribing regulatory sequences will include a promoter region which includes a promoter sequence for transcriptional control of the operably joined gene. Regulatory sequences may also include enhancer sequences or upstream activator sequences, as desired.

“Regulation” and “regulate” refer to the modulation of gene expression controlled by DNA sequence elements located primarily, but not exclusively upstream of (5′ to) the transcription start of a gene. Regulation may result in an all or none response to stimulation, or it may result in variations in the level of gene expression.

In a further aspect, the invention concerns a host cell transformed with the expression vector according to the invention.

“Cells”, “host cells” or “recombinant host cells” are terms used interchangeably herein. It is understood that such terms refer not only to the particular subject cells but to the progeny or potential progeny of such a cell. Because certain modification may occur in succeeding generation due to either mutation or environmental influences, such progeny may not, in fact, be identical to the parent cell, but are still included within the scope of the term as used herein.

“Host cell” as used herein refers to cells which can be recombinantly transformed with naked DNA or expression vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival.

The host cells of the invention are transformed or transfected with the expression vector descried herein to express the synthetic spider silk protein of the invention. “Transformation”, as used herein, refers to a process in which a cell's genotype is changed as a result of the cellular uptake of exogenous DNA or RNA, and, for example, the transformed cell expresses a recombinant form of the desired synthetic spider silk protein. The term “transfection” means the introduction of a nucleic acid, e.g., naked DNA or an expression vector, into a recipient cells by nucleic acid-mediated gene transfer.

In one specific embodiment, the host cells transformed with the expression vector according to the invention are insect cells. As insect cells, Lepidoptera insect cells may be used, more specifically cells from Spodopterafrugiperda and from Trichoplusiani. Most specifically, the insect cell is a Sf9, Sf21 or high 5 cells.

In some embodiments, the silk protein of the invention is devoid of post translational modifications.

In some embodiments, the silk protein of the invention is biodegradable. This characteristic may be of importance, for example, in the field of medicine, whenever the silk proteins are intended for an in vivo use, in which biological degradation is desired. This characteristic may in particular find application in suture materials and wound closure and coverage systems.

According to some aspects, the invention concerns an expression vector comprising the nucleic acid sequence of the present invention, wherein the nucleic acid sequence is under expression control of an operably linked promoter and, optionally, regulatory sequences.

In some embodiments, the mixture of proteins results in a self-assembled forming a defined structure. In some embodiments, the mixture of proteins is in the form of a network. In some embodiments, the mixture of proteins is in the form of a complex. In some embodiments, the mixture of proteins induces a defined secondary structure, e.g., a beta turn, gamma turn, beta sheet, alpha helix conformation, and the like.

According to some aspects, the disclosed mixture of proteins is in the form of a fiber. A “fiber” as used herein, is meant a fine cord of fibrous material composed of two or more filaments twisted together. By “filament” is meant a slender, elongated, threadlike object or structure of indefinite length, ranging from microscopic length to lengths of a mile or greater. Specifically, the synthetic spider silk filament is microscopic, and is proteinaceous. By “biofilament” is meant a filament created from a protein, including recombinantly produced spider silk protein. In some embodiments, the term “fiber” does not encompass unstructured aggregates or precipitates.

In some embodiments, the fiber of the proteins is characterized by size of at least one dimension thereof (e.g., diameter, length). For example, and without limitation, the diameter of the fiber is between 10 nm-1 μm, 20-100 nm, or 10-50 nm.

In some embodiments, the fiber is composed of nano-fibrils. In some embodiments, the nano-fibrils have a diameter of e.g., 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24 nm, about 25 nm, about 26 nm, about 27 nm, about 28 nm, about 29 nm, about 30 nm, about 31 nm, about 32 nm, about 33 nm, about 34 nm, about 35 nm, about 36 nm, about 37 nm, about 38 nm, about 40 nm, about 42 nm, about 44 nm, about 46 nm, about 48 nm, or about 50 nm, including any value or range therebetween. In one embodiment, the nano-fibrils have a diameter of 3-7 nm. In one embodiment, the nano-fibrils have a diameter of 4-6 nm.

In some embodiments, the length of the disclosed fiber is between 1-200 μm, 10-100 μm, 100 to 500 μm or 200-500 μm.

In some embodiments of any one of the embodiments described herein, the disclosed fiber is characterized by a porous structure. In some embodiments, the porous structure is characterized by a porosity of at least 30% (e.g., from 30 to 99%). In some embodiments, the porous structure is characterized by a porosity of at least 50% (e.g., from 50 to 99%). In some embodiments, the porous structure is characterized by a porosity of at least 60% (e.g., from 60 to 99%). In some embodiments, the porous structure is characterized by a porosity of at least 70% (e.g., from 70 to 99%). In some embodiments, the porous structure is characterized by a porosity of at least 80% (e.g., from 80 to 99%). In some embodiments, the porous structure is characterized by a porosity of at least 90% (e.g., from 90 to 99%). In some embodiments, the porous structure is characterized by a porosity of about 90%.

Herein, the term “porosity” refers to a percentage of the volume of a substance (e.g., a “sponge-like” material) which consists of voids. In another embodiment, porosity is measured according to voids within the surface area divided to the entire surface area (porous and non-porous).

In some embodiments, the porous structure of the disclosed fibers allows absorbing water efficiently on the fiber surface. That is, and without being bound by any particular theory, this surprising discovery can be explained in view of the disclosed fiber structure and its porosity which is in sharp distinction from native spider silk found in nature.

In some embodiments of any one of the embodiments described herein, the disclosed fiber is characterized by a mean diameter is nanosized.

In some embodiments, the disclosed fiber is characterized by a mean diameter is in a range of from 1 to 50 nm. In some such embodiments, the mean diameter is in a range of from 3 to 50 nm. In some such embodiments, the mean diameter is in a range of from 5 to 50 nm. In some such embodiments, the mean diameter is in a range of from 1 to 40 nm. In some such embodiments, the mean diameter is in a range of from 1 to 30 nm. In some such embodiments, the mean diameter is in a range of from 5 to 40 nm.

As further exemplified in the Examples section below, in some embodiments, a plurality of the disclosed fibers may be in the form of self-assembled structure or matrix. In some embodiments, this matrix can be rendered suitable for biomaterial applications.

In some embodiments, a fiber or a mixture of proteins comprises “m” types of proteins of differing molecular weight, wherein each protein in the “m” types of proteins comprises, independently, “n” repeats of a repetitive region of a major ampullate spidroin (MaSp) protein, or a functional homolog, variant, derivative or fragment thereof, wherein m is an integer between 2 to 70 and n is an integer between 6 to 70. In some embodiments, a fiber or a mixture of proteins comprises “m” types of proteins of differing molecular weight, wherein each protein in the “m” types of proteins comprises, independently, “n” repeats of a repetitive region of a major ampullate spidroin (MaSp) protein, or a functional homolog, variant, derivative or fragment thereof, wherein m is an integer between 2 to 70 and n is an integer between 7 to 70. In some embodiments, a fiber or a mixture of proteins comprises “m” types of proteins of differing molecular weight, wherein each protein in the “m” types of proteins comprises, independently, “n” repeats of a repetitive region of a major ampullate spidroin (MaSp) protein, or a functional homolog, variant, derivative or fragment thereof, wherein m is an integer between 2 to 70 and n is an integer between 8 to 70. In one embodiment “n” repeats of a repetitive region of a major ampullate spidroin (MaSp) protein must be equal or greater than 6 in order to efficiently support cell growth, cell expansion and proliferation, multi-layer cell assembly, cell migration, reduced cell death, tissue regeneration and/or healing processes. In one embodiment “n” repeats of a repetitive region of a major ampullate spidroin (MaSp) protein must be equal or greater than 7 in order to efficiently support cell growth, cell expansion and proliferation, multi-layer cell assembly, cell migration, reduced cell death, tissue regeneration and/or healing processes. In one embodiment “n” repeats of a repetitive region of a major ampullate spidroin (MaSp) protein must be equal or greater than 8 in order to efficiently support cell growth, cell expansion and proliferation, multi-layer cell assembly, cell migration, reduced cell death, tissue regeneration and/or healing processes. In one embodiment “n” repeats of a repetitive region of a major ampullate spidroin (MaSp) protein must be equal or greater than 9 in order to efficiently support cell growth, cell expansion and proliferation, multi-layer cell assembly, cell migration, reduced cell death, tissue regeneration and/or healing processes. In one embodiment “n” repeats of a repetitive region of a major ampullate spidroin (MaSp) protein must be equal or greater than 10 in order to efficiently support cell growth, cell expansion and proliferation, multi-layer cell assembly, cell migration, reduced cell death, tissue regeneration and/or healing processes.

In some embodiments, one or more fibers in the disclosed matrix comprise at least 6, at least 7, least 8, at least 9, at least 10, at least 11, or at least 12, repeats (“n” as defined hereinabove). In some embodiments, one or more fibers in the disclosed matrix comprise are: 6-70, 7, 8-70, 9-70, 10-70, 11-70, 12-70, 13-70, 14-70, 15-70, 16-70, 17-70, 18-70, 19-70, or 20-70 repeats (“n” as defined hereinabove). In some embodiments, the cell, medical and biological compositions and methods as described herein require at least 6 repeats. In some embodiments, the cell, medical and biological compositions and methods as described herein require at least 7 repeats. In some embodiments, the cell, medical and biological compositions and methods as described herein require at least 8 repeats. In some embodiments, the cell, medical and biological compositions and methods as described herein require: 6-70, 7, 8-70, 9-70, 10-70, 11-70, 12-70, 13-70, 14-70, 15-70, 16-70, 17-70, 18-70, 19-70, or 20-70 repeats (“n” as defined hereinabove).

In some embodiments, this matrix is suitable for cell growth, and for maintaining or promoting cellular activity, as further demonstrated hereinbelow.

In some embodiments, the term “self-assembled” refers to a resulted structure of a self-assembly process (e.g., spontaneous self-assembly process) based on a series of associative chemical reactions between at least two domains of the fiber(s), which occurs when the associating groups on one domain are in sufficient proximity and are oriented so as to allow constructive association with another domain. In other words, an associative interaction means an encounter that results in the attachment of the domains of a fiber or fibers to one another. In some embodiments, attached domains are not parallel to each other. Also contemplated are arrangements in which there are more than two domains of the self-assembled structure, each engaging a different plane.

It is noteworthy, that in some embodiments, the density of the self-assembled fiber (e.g., about 80% voids) is in the range of from 0.1 g/cm³ to 0.4 g/cm³ or from 0.2 g/cm³ to 0.3 g/cm³. In exemplary embodiments, the density of the self-assembled fiber is about 0.26 g/cm³.

Wettability study of surfaces at nano-scale spatial resolution and high temporal resolution is an emerging field from both theoretical and practical aspects.

As demonstrated in the Examples section that follows, the disclosed fiber exhibited a high degree of surface's wettability exhibiting a remarkable ability to absorb fluids in comparison with their volume and weight.

In some embodiments, the polymer is hydrophobic. In some embodiments the polymer is UV cured.

In some embodiments, the disclosed composite is biostable. In some embodiments, the disclosed composite is biocleavable. In some embodiments, the disclosed composite is biodegradable.

In some embodiments, the term “biostable” describes a compound or a polymer that remains intact under physiological conditions (e.g., is not degraded in vivo, and hence is non-biodegradable or non-biocleavable).

In some embodiments, the term “biodegradable” describes a substance which can decompose under physiological and/or environmental condition(s) into breakdown products. Such physiological and/or environmental conditions include, for example, hydrolysis (decomposition via hydrolytic cleavage), enzymatic catalysis (enzymatic degradation), and mechanical interactions. This term typically refers to substances that decompose under these conditions such that 50 weight percent of the substance decompose within a time period shorter than one year.

In some embodiments, the term “biodegradable” as used in the context of embodiments of the invention, also encompasses the term “bioresorbable”, which describes a substance that decomposes under physiological conditions to break down products that undergo bioresorption into the host-organism, namely, become metabolites of the biochemical systems of the host-organism.

### General:

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a” “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

The current application makes use of the following methods: solvent casting and extrusion. The following materials were enriched: PCL and Polypropylene (PP).

### Pretreatment of Spider Silk (SVX) Fibers

An aliquot (according to a w/w ratio relative to the polymer weight) of an aqueous suspension of SVX fibers (“SVX milk”) was added to a tube. The suspension was made homogenic by shaking, vortexing and observing to detect aggregates on the tube walls, so as to obtain homogenous “SVX milk”. “SVX milk” was centrifuged for 5 min/5200 g.

The aqueous supernatant (should be transparent with some white colloidal particles) was discarded. The pellet was broken and 100% EtOH was added little by little (about 30% of the pellet volume on each addition) till reaching volume equivalent to that of the pellet. The mixture was mixed to reach homogeneity.

Gradually the volume was adjusted to 10 ml with 100% EtOH, with continue mixing to ensure homogeneity. 100% EtOH was added to a final volume that equals 10 times the initial pellet volume. The SVX fibers were resuspended to homogeneity, including visual verification of the tube walls.

The solution was filtered through a 40 μm filter followed by centrifugation for 4 min/4500 g. The abovementioned steps were repeated by replacing EtOH with dry THF, followed by dry Toluene, and by dry Xylene, thereby obtaining pretreated SVX fibers.

In-Situ Polymerization of PCL with SVX Fibers

Under nitrogen atmosphere, 200 μl triethylamine were added to a solution of Xylene containing 100 mg of pretreated fibers. 400 μl of ε-Caprolactone were then added. After warming the solution to the bath temperature (135° C.), 50 μl Tin (11) 2-ethylhexanoate was added. The mixture was left stirring in the heating bath for 72 hours. The excess of unreacted materials was washed at the end of the reaction following by drying or evaporating of the residual solvent, so as to obtain modified SVX fibers.

Preparation of a PCL Solution Enriched with Modified Fibers

To a 1 g of PCL a sufficient amount of the modified SVX fibers (in-situ polymerized PCL-SVX or PLA-SVX fibers) was added together with 10 mL of Toluene. The mixture was stirred for 6 hours. Once the solution is ready, the entire polymer dissolves. The stirring was turned off and the solution was degassed for approximately two hours.

### Solution Casting and Evaporation

The solution was added to a clean Petri dish (in a hood) and left to evaporate for 12 hours.

### Example 1

Mechanical Properties of PCL Films Enriched with SVX-PCL Fibers

PCL was solubilized in an organic solvent, followed by addition of SVX fibers modified with caprolactone (SVX-PCL) and silicone surfactant (BYK). The mixture was then solvent cast it into films and their mechanical properties were measure and are presented in Table 1. Pristine (i.e. without any additives) PCL was utilized as a control.

TABLE 1

| | | | | Upper | Stress at | Stress at | | Total | Work |
|---|---|---|---|---|---|---|---|---|---|
| | | | Young's | Yield | 30% | 100% | Tensile | Elongation | of |
| | Thickness | Area | Modulus | Strength | Modulus | Modulus | Strength | at Fracture | Failure |
| | (mm) | (mm²) | (MPa) | (MPa) | (MPa) | (MPa) | (MPa) | (%) | (Nmm) |
| Control | 0.105 | 0.72975 | 347.57 | 15.953 | 13.643 | 13.536 | 25.895 | 659.45 | 2545.5 |
| | 0.11 | 0.7645 | 320.74 | 15.411 | 13.493 | 13.29 | 33.994 | 936.88 | 4588.1 |
| | 0.105 | 0.72975 | 376.3 | 16.401 | 13.897 | 13.771 | 20.521 | 480.42 | 1636.1 |
| 6% | 0.13 | 0.897 | 362.45 | 14.224 | 10.798 | 11.073 | 17.254 | 543.7 | 1876 |
| [BYK] | 0.145 | 1.015 | 364.46 | 14.042 | 11.718 | 11.936 | 18.507 | 584.96 | 2351.1 |
| | 0.145 | 0.9976 | 358.97 | 13.317 | 10.722 | 11.211 | 18.598 | 603.93 | 2386 |

### Example 2

Mechanical Properties of PCL Films Enriched with SVX-PLA Fibers

PCL was solubilized in an organic solvent, followed by addition of SVX fibers modified with PLA (SVX-PLA). The mixture was then solvent cast it into films and their mechanical properties were measure and are presented in Table 2. Pristine (i.e. without any additives) PCL was utilized as a control.

TABLE 2

| | | | | Upper | Stress at | Stress at | | Percentage | Work |
|---|---|---|---|---|---|---|---|---|---|
| | | | Young's | Yield | 30% | 100% | Tensile | Total | of |
| | Thickness | Area | Modulus | Strength | Modulus | Modulus | Strength | Elongation | Failure |
| | (mm) | (mm²) | (MPa) | (MPa) | (MPa) | (MPa) | (MPa) | at Fracture | (Nmm) |
| Control | 0.101 | 0.702 | 251.29 | 16.437 | 11.83 | 11.911 | 23.479 | 611.06 | 2024.4 |
| | 0.098 | 0.681 | 296.94 | 15.136 | 12.561 | 12.847 | 26.182 | 657.58 | 2311.2 |
| | 0.103 | 0.736 | 322.09 | 14.357 | 11.965 | 12.278 | 21.321 | X | 2656.4 |
| | 0.098 | 0.647 | 301.92 | 22.499 | 13.284 | 13.387 | 22.499 | 580.25 | 1786.2 |
| | 0.098 | 0.689 | 320.55 | 16.047 | 13.392 | 13.233 | 18.754 | 458.16 | 1386.1 |
| | 0.1 | 0.673 | 327.3 | 15.328 | 12.792 | 13.272 | 18.062 | 455.75 | 1293.6 |
| 4% | 0.107 | 0.75114 | 285.21 | 18.556 | 10.126 | 10.434 | 18.556 | 521.89 | 1520.6 |
| SVX- | 0.105 | 0.74025 | 363.26 | 14.561 | 12.288 | 13.895 | 17.055 | 396.12 | 1171 |
| PLA | 0.107 | 0.749 | 341.96 | 14.622 | 13.036 | 12.729 | 15.512 | 401.98 | 1156.2 |
| | 0.105 | 0.7266 | 380.04 | 14.997 | 13.549 | 12.263 | 16.539 | 427.18 | 1225.7 |
| | 0.11 | 0.7777 | 334.15 | 14.187 | 12.656 | 11.91 | 16.673 | 432.85 | 1336.7 |

### Example 3

Mechanical Properties of PCL Threads (Sutures) Enriched with SVX-PCL Fibers

PCL was solubilized in an organic solvent, followed by addition of SVX fibers modified with polycaprolactone (PCL) (SVX-PCL). The mixture was then solvent cast into films which were used as feed stock material for melt extrusion of the composite material into fine threads of about 0.5 mm in diameter. The extruded threads were later drawn to 10 times their original length using a tensiometer, reaching a diameter of about 0.2 mm. The mechanical properties of these threads were evaluated and were compared to a degradable suture benchmark (used as a control). The enriched sutures of the invention exhibited increased Young's modulus, increased elasticity (decreased hysteresis), increased knot strength together with a significantly lower degradation rate, compared to the control (see FIGS. 4-7).

### Example 4

Mechanical Properties of Polypropylene (PP) Threads (Sutures) Enriched with SVX-PCL Fibers

PP was solubilized in an organic solvent, followed by addition of SVX fibers polymerized with polycaprolactone (PCL) (SVX-PCL). The mixture was then solvent cast into films which were used as feed stock material for melt extrusion of the composite material into fine threads of about 0.5 mm in diameter. The extruded threads were later drawn to 10 times their original length using a tensiometer, reaching a diameter around 0.2 mm. The mechanical properties of these threads were evaluated and were compared to a non-degradable polypropylene suture benchmark (used as a control). The enriched sutures of the invention exhibited increased Young's and increased elasticity (decreased hysteresis), compared to the control (see FIGS. 8-10).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A composition comprising a copolymer, wherein said copolymer comprises a porous ampullate spidroin protein (MaSp)-based fiber covalently bound to a first polymer.

2. The composition of claim 1, wherein said copolymer is in a form of a graft-copolymer and wherein said bound is via an amino acid of said MaSp-based fiber.

3. The composition of claim 2, wherein said amino acid is selected from the group consisting of tyrosine, serine, lysine, and cysteine or any combination thereof.

4. The composition of claim 1, further comprising a second polymer having a molecular weight in the range of 1000 Da to 1000 kDa, optionally wherein said second polymer comprises a partially branched polymer; and wherein a weight per weight (w/w) ratio of the first polymer to the second polymer within said composition is between 1:7 and 1:30.

5. The composition of claim 1, wherein said copolymer is present at a concentration of 0.1% to 30%, by total weight of said composition.

6. The composition of claim 4, wherein said first polymer and said second polymer are each independently selected from a synthetic polymer, a thermoplastic polymer, a thermoset an epoxy, a polyester a polyamide, a polyol, a polyurethane, polyethylene, Nylon, a polyacrylate, a polycarbonate, polylactic acid (PLA) or a copolymer thereof a silicon, a liquid crystal polymer, a maleic anhydride grafted polypropylene, polycaprolactone (PCL), rubber, cellulose, or any combination thereof; and wherein a w/w ratio of the first polymer to the MaSp-based fiber is between 10:1 and 1:1.

7. The composition of claim 1, wherein said first polymer is biodegradable, and has a molecular weight in the range of 100 Da to 1000 kDa.

8. The composition of claim 1, wherein said MaSp-based fiber comprises a mixture of proteins comprising “m” types of proteins of differing molecular weight, wherein each protein in said mixture comprises, independently, “n” repeats of a repetitive region of a MaSp protein, or a functional homolog, variant, derivative or fragment thereof, wherein m and n are, independently, an integer between 2 to 70; optionally wherein said mixture of proteins is characterized by one or more properties selected from the group consisting of: a. each repeat has a molecular weight in the range of 2 kDa to 3.5 kDa; b. the ratio of‘n’ to ‘m’ is in the range of 1.5:1 to 1:1.5.

9. The composition of claim 8, wherein each of said proteins comprise, independently, an amino acid sequence as set forth in SEQ ID NO: 1:
(X₁)ZX2GPGGYGPX3X4X5GPX6GX7GGX8GPGGPGX9X10,
wherein X₁ is, independently, at each instance A or G wherein at least 50% of (X₁)Z is A, Z is an integer between 5 to 30; X₂ is S or G; X₃ is G or E; X₄ is G, S or N; X₅ is Q or Y; X₆ is G or S; X₇ is P or R; X₈ is Y or Q; X₉ is G or S; and X₁₀ is S or G; optionally wherein said repetitive region comprises the amino acid sequence as set forth in SEQ ID NO: 3 (AAAAAAAASGPGGYGPGSQGPSGPGGYGPGGPGSS).

10. The composition of claim 1, wherein said MaSp-based fiber is characterized by a porosity of at least 30%; optionally wherein the composition further comprises a third polymer; further optionally wherein a w/w content of said third polymer is 30% to 99% of the total composition.

11. An article comprising the composition of claim 1; and wherein said article is in a form of a thread, a sheet or a film.

12. The article of claim 11, comprising a biocompatible material, and optionally comprising a coating; wherein said article is in a form of a suture, surgical mesh, medical adhesive strips, electrospun mesh, skin grafts, fat grafts, cosmetics, dermal fillers, drug eluting/delivery device, replacement ligaments, clothing fabric, bullet-proof vest lining, cable, tube, film, rope, fishing line, tires, sports equipment, and reinforced plastics.

13. The article of claim 11, being in a form of a surgical suture and comprising PCL enriched with said copolymer, wherein the first polymer comprises the MaSp-based fiber covalently bound to PCL; optionally wherein said enriched is between 0.1% and 30%, by total weight of said article.

14. The article of claim 13, wherein said wherein said article is a medical device having a Young's modulus of at least 600 MPa; optionally wherein the article is a surgical suture.

15. A method for synthesizing the composition of claim 1 comprising: mixing a major ampullate spidroin protein (MaSp)-based fiber with a monomer under conditions suitable for said monomer to polymerize, thereby forming an in-situ polymerized first polymer bound to said MaSp-based fiber via a covalent bond.

16. The method of claim 15, wherein said polymerize is via a ring-opening polymerization; wherein said conditions comprise a time period between 1 hour and 4 days and a temperature between 2° and 200° C.; and wherein a w/w ratio of said monomer to said MaSp-based fiber is between 1:1 and 10:1.

17. The method of claim 15, wherein said method further comprises mixing said first polymer bound to said MaSp-based fiber with a second polymer; wherein a w/w ratio of said second polymer to said first polymer bound to said MaSp-based fiber is between 100:1 to 5:1.

18. The method of claim 17, wherein said second polymer is selected from a synthetic polymer, a thermoplastic polymer, a thermoset an epoxy, a polyester, a polypropylene, a polyethylene, a polyamide, a polyol, a polyurethane, polyethylene, Nylon, a polyacrylate, a polycarbonate, polylactic acid (PLA) or a copolymer thereof a silicon, a liquid crystal polymer, a maleic anhydride grafted polypropylene, polycaprolactone (PCL), rubber, cellulose, or any combination thereof.

19. The method of claim 15, wherein said monomer comprises a lactone.


SEQUENCE LISTING (13 sequences)

SEQ ID NO: 1 - protein, 28 aa, synthetic construct
  REGION 1..28: note = synthetic
  SITE 1: note = MISC_FEATURE - X is A or G
  SITE 2: note = MISC_FEATURE - X is S or G
  SITE 10: note = MISC_FEATURE - X is G or E
  SITE 11: note = MISC_FEATURE - X is G, S or N
  SITE 12: note = MISC_FEATURE - X is Q or Y
  SITE 15: note = MISC_FEATURE - X is G or S
  SITE 17: note = MISC_FEATURE - X is P or R
  SITE 20: note = MISC_FEATURE - X is Y or Q
  SITE 27: note = MISC_FEATURE - X is G or S
  SITE 28: note = MISC_FEATURE - X is S or G
XXGPGGYGPXXXGPXGXGGXGPGGPGXX

SEQ ID NO: 2 - protein, 27 aa, synthetic construct
  REGION 1..27: note = synthetic
SGPGGYGPGSQGPSGPGGYGPGGPGSS

SEQ ID NO: 3 - protein, 35 aa, synthetic construct
  REGION 1..35: note = synthetic
AAAAAAAASGPGGYGPGSQGPSGPGGYGPGGPGSS

SEQ ID NO: 4 - protein, 35 aa, synthetic construct
  REGION 1..35: note = synthetic
AAAAAAAASGPGGYGPENQGPSGPGGYGPGGPGSS

SEQ ID NO: 5 - protein, 40 aa, synthetic construct
  REGION 1..40: note = synthetic
MSYYHHHHHHDYDIPTTENLYFQGAMDPEFKGLRRRAQLV

SEQ ID NO: 6 - protein, 50 aa, synthetic construct
  REGION 1..50: note = synthetic
MSYYHHHHHHDYDIPTTENLYFQGAMDPEFKGLRRRAQLVRPLSNLDNAP

SEQ ID NO: 7 - protein, 54 aa, synthetic construct
  REGION 1..54: note = synthetic
MSYYHHHHHHDYDIPTTENLYFQGAMDPEFKGLRRRAQLVDPPGCRNSARAGSS

SEQ ID NO: 8 - protein, 97 aa, synthetic construct
  REGION 1..97: note = synthetic
  SITE 1: note = MISC_FEATURE - X is A or G
  SITE 2: note = MISC_FEATURE - X is S or G
  SITE 11: note = MISC_FEATURE - X is S or G
  SITE 12: note = MISC_FEATURE - X is Q or Y
  SITE 15: note = MISC_FEATURE - X is S or G
  SITE 17: note = MISC_FEATURE - X is P or R
  SITE 20: note = MISC_FEATURE - X is Y or Q
  SITE 27: note = MISC_FEATURE - X is G or S
  SITE 28: note = MISC_FEATURE - X is G or S
VAASRLSSPAASSRVSSAVSSLVSSGPTNGAAVSGALNSLVSQISASNPGLSGCDALVQALLELVSALVAILSSASIGQVNVSSVSQSTQMISQALS

SEQ ID NO: 9 - protein, 114 aa, synthetic construct
  REGION 1..114: note = synthetic
GPSGPGAYGPSPSASASVAASRLSSPAASSRVSSAVSSLVSSGPTNGAAVSGALNSLVSQISASNPGLSGCDALVQALLELVSALVAILSSASIGQVNVSSVSQSTQMISQALS

SEQ ID NO: 10 - protein, 555 aa, synthetic construct
  REGION 1..555: note = synthetic
AAAAAAASGSGGYGPENQGPSGPVAYGPGGPVSSAAAAAAAGSGPGGYGPENQGPSGPGGYGPGGSGSSAAAAAAAASGPGGYGPGSQGPSGPGGSGGYGPGSQGPSGPGASSAAAAAAAASGPGGYGPGSQGPSGPGAYGPGGPGSSAAASGPGGYGPGSQGPSGPGGSGGYGPGSQGPSGPGGPGASAAAAAAAAASGPGGYGPGSQGPSGPGAYGPGGPGSSAAASGPGGYGPGSQGPSGPGAYGPGGPGSSAAAAAAAGSGPGGYGPGNQGPSGPGGYGPGGPGSSAAAAAAASGPGGYGPGSQGPSGPGVYGPGGPGSSAAAAAAAGSGPGGYGPGNQGPSGPGGYGPGGSGSSAAAAAAAASGPGGYGPGSQGPSGPGGSGGYGPGSQGPSGPGASSAAAAAAAASGPGGYGPGSQGPSGPGAYGPGGPGSSAAASGPGGYGPGSQGPSGPGAYGPGGPGSSAAAAAAASGPGGYGPGSQGPSGPGGSRGYGPGSQGPGGPGASAAAAAAAAASGPGGYGPGSQGPSGPGYQGPSGPGAYGPSPSASAS

SEQ ID NO: 11 - protein, 6 aa, synthetic construct
  REGION 1..6: note = synthetic
HHHHHH

SEQ ID NO: 12 - protein, 9 aa, synthetic construct
  REGION 1..9: note = synthetic
YPYDVPDYA

SEQ ID NO: 13 - protein, 11 aa, synthetic construct
  REGION 1..11: note = synthetic
MASMTGGQQMG